SEQUENCE LISTING (9 sequences)

SEQ ID NO: 1 - protein, 72 aa, Artificial Sequence
  Var24
DAEFRHDSGYEVFNNFTVSFWLRVPKVSASHLEDAEFRHDSGYEVQYIKANSKFIGITELDAEFRHDSGYEV

SEQ ID NO: 2 - protein, 73 aa, Artificial Sequence
  Met-Var24
MDAEFRHDSGYEVFNNFTVSFWLRVPKVSASHLEDAEFRHDSGYEVQYIKANSKFIGITELDAEFRHDSGYEV

SEQ ID NO: 3 - protein, 73 aa, Artificial Sequence
  X-Var24
  Xaa (1)..(1): X = an amino acid, preferably a natural amino acid
XDAEFRHDSGYEVFNNFTVSFWLRVPKVSASHLEDAEFRHDSGYEVQYIKANSKFIGITELDAEFRHDSGYEV

SEQ ID NO: 4 - protein, 15 aa, Artificial Sequence
  P2
QYIKANSKFIGITEL

SEQ ID NO: 5 - protein, 21 aa, Artificial Sequence
  P30
FNNFTVSFWLRVPKVSASHLE

SEQ ID NO: 6 - protein, 453 aa, Artificial Sequence
  BS aducanumab HC
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 214 aa, Artificial Sequence
  BS aducanumab LC
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, Artificial Sequence
  BS bapineuzumab HC
EVQLVESGGGLVQPGGSLRLSCAGSGFTFSNYGMSWVRQAPGKGLEWVASIRSGGGRTYYSDNVKGRFTISRENAKNSLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 219 aa, Artificial Sequence
  BS bapineuzumab LC
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0275125 A1
(43) Pub. Date: Sep. 12, 2019

(54) ABETA VARIANTS, ASSAY, METHOD AND TREATMENT OF ALZHEIMER'S DISEASE

(71) Applicant: H. Lundbeck A/S, Valby (DK)

(72) Inventors: Lars Østergaard Pedersen, Valby (DK); Dorte Kornerup Ditlevsen, Valby (DK)

(21) Appl. No.: 16/295,877

(22) Filed: Mar. 7, 2019

(30) Foreign Application Priority Data
Mar. 9, 2018 (DK) PA201800109

(51) Int. Cl. A61K 39/00; A61P 25/28; A61K 9/00; C07K 14/33; C07K 14/47; A61K 39/08; G01N 33/68

(52) U.S. Cl. CPC A61K 39/0005; A61P 25/28; A61K 9/0019; C07K 14/33; A61K 2039/545; A61K 39/08; G01N 33/6896; A61K 2039/54; C07K 14/4711

(57) ABSTRACT

The present invention relates to a new abeta immunogen variants that enables efficient treatment of Alzheimer's Disease patients by raising specific antibodies against oligimeric and toxic abeta deposits in the brain of Alzheimer's patients. The invention also relates to an assay that enables efficient treatment of Alzheimer's Disease patients by assessing and monitoring the titre response to active immune therapy, as well as treatment and identification of specific subpopulations of Alzheimer Disease patients

### CROSS-REFERENCE TO RELATED APPLICATION

This application claims priority to Danish Application No. PA201800109, filed Mar. 9, 2018, the entire contents of which is hereby incorporated herein by reference it its entirety.

### FIELD OF THE INVENTION

The present invention relates to new abeta immunogen variants that enables efficient treatment of Alzheimer's Disease patients by raising specific antibodies against oligomeric and toxic abeta deposits in the brain of Alzheimer's patients. The invention also relates to an assay that enables efficient treatment of Alzheimer's Disease patients by assessing and monitoring the titre response to active immune therapy, as well as treatment and identification of specific subpopulations of Alzheimer Disease patients.

### BACKGROUND OF THE INVENTION

Amyloid beta (abeta) denotes peptides with a length of between 1-39 to 1-43 amino acids that are involved in Alzheimer's disease (AD). Cerebral and cerebro-vascular accumulation of abeta, is widely accepted as the key neuropathological event in AD. AD main features are in fact amyloid plaques, mainly formed by abeta aggregates, and abeta deposition at vessel level, also referred to as Cerebral Amyloid Angiopathy, deriving by proteolysis of the amyloid precursor protein (APP) by beta- and gamma-secretase.

Various mechanisms seem to be accountable for the formation of these accumulations, among which are malfunctioning of toxic aggregate clearance systems or malfunctioning of cell defence systems at cerebral level. Therapeutic approaches defined as “Amyloid-modifying therapies” and in particular those founded on active and passive immunisation, are based precisely on a possible restoration and potentiation of these mechanisms.

Passive immunotherapy against abeta in AD patients have been tested in clinical trials. Tested antibodies such as aducanumab and bapineuzumab bind N-terminal epitopes of abeta and represent two very different therapeutically outcome.

Aducanumab bind abeta at the first 3-9 amino acids, but only in abeta in oligomerized and aggregated format and has no or barely detectable binding to monomer abeta forms. Aducanumab has shown promising effects in clinical trial by showing reduced abeta load and significant beneficial effects. It is commonly thought that soluble abeta oligomers, rather than monomers or plaques, may be the primary toxic species. Considering that abeta plaques might be a source of abeta oligomers, treatment with aducanumab might slow release of abeta oligomers by the plaques and thereby limiting their toxic effect on neurons. In fact, chronic dosing of 18-month old Tg2576 transgenic mice with aducanumab led to normalization of neuritic calcium overload in the brain. Other studies have linked calcium dyshomeostasis in neurons and microglia to binding of abeta oligomers to metabotropic receptors. Aducanumab binding to soluble abeta oligomers may prevent their interaction with those receptors, thereby preventing the detrimental effect of membrane depolarization. Restoration of this functional endpoint suggests that aducanumab treatment may lead to beneficial effects on neuronal network function underlying cognitive deficits (Jeff Sevigny et al, 2016, Nature, p 50-56).

Bapineuzumab bind abeta amino acid 1-5 and both mono, oligomer and aggregated forms of abeta with high affinity. Bapineuzumab did not improve clinical outcomes in patients with Alzheimer's disease, despite treatment differences in biomarkers observed in APOE c4 carriers. Some safety findings were reported among patients receiving bapineuzumab (Salloway et al, N Engl J Med 2014; 370:322-33).

The inventors of the present invention disclose new immunogen variants (Met-var24 and X-var24) containing an additional amino acid (such as methionine) in the N-terminal part of the molecule, which alters the properties of the induced antibody response in an unforeseeable way and provide new functional advantages. In particular, the variants enable binding of adacuanumab, which so far is the only clinical successful antibody candidate binding to specific oligomeric toxic forms of abeta and further provides evidence for the variants can induce the generation of aducanumab-like antibodies.

That the N-terminal methionine plays an important role for the antibody response was not foreseen, because many immunogens and vaccines tend to avoid this “foreign” amino acids and tempt to mimic the natural version as much as possible.

Removal of the translation initiator N-formyl-methionine or methionine from a recombinant pharmaceutical protein is often critical for its function and stability as seen in, for example, human hemoglobin, interleukin-2 or growth hormones (Busby Jr. et al. 1987, J. Biol. Chem. 262: 8532-8536; Boix et al. 1996, J. Mol. Biol. 257: 992-1007; Varshaysky 1996. Proc. Natl. Acad. Sci. 93: 12142-12149; Adachi et al. 2000. Protein Expr. Purif. 20: 37-44; Endo et al. 2001. Biochemistry 40: 914-919).

The incorporation of a methionine residue at the N-terminal end of a polypeptide is part of the translational initiation signal in E. coli and other prokaryotic cells. However, the methionine residue carried by the initiator tRNA is N-formylated prior to incorporation. Once made, many bacterial proteins are subjected to post-translational modification reactions which remove sequentially the formyl group and the terminal methionine residue in E. coli so that in E. coli only a portion of the polypeptide chains found in the cytoplasm retain their methionine. In cytosolic extract of E. coli only 40% of the polypetidic chains retain an N-terminal methionine. Instead, about 50% display alanine, serine or threonine at their N-termini. Some investigations indicate that extend of methionine removal seems to be dependent on the side chain length of the penultimate amino acid (Hirel et al., Proc. Natl. Acad. Sci., Vol 86, p 8247-8251).

Of importance is that foreign genes expressed in E. coli has less tendency to have methionine removed. These polypeptides having an addition of an N-terminal methionine will not be identical to the naïve human sequence due to the additional methionine. One such product, methionyl (human) growth hormone has been used clinically. However, the potential immunogenicity of modified human proteins, considerable efforts have been made to produce therapeutic products without the terminal methionine, and several different strategies can be used.

One strategy is to employ a vector which uses methionine as a link that can be specifically cleaved between the recombinant polypeptide and a bacterial carrier peptide, producing an end product without N-terminal methionine. This method has been used in producing insulin, wherein cyanogen bromide was used to cleave the polypeptides chains at a methionine residue.

Another strategy can be to use part of the E. coli protein export system to produce an rDNA-derived product that is not only free of N-terminal methionine, but also exported into the periplasmic space. Many bacterial secreted proteins are synthesized as precursor proteins which contains an additional hydrophobic N-terminal signal sequence which is cleaved during the passage through the membrane. This system has been used to produce human growth hormone without terminal methionine using E. coli. (Polypeptide Protein Drugs, R. Hider and D. Barlow, Ellis Horwood Ltd, 1991, page 91-92)

A further, strategy which has been reported efficient can be to use recombinant methionine aminopeptidase to reduce the N-terminal methionine of E. coli produced polypeptides (Liao Y D et al., Protein Sci. 2004; 13:1802-1810; Shapiro et al. 1988. Anal. Biochem. 175: 450-461; Notomista et al. 1999. FEBS Lett. 463: 211-215). Proteases may also be used, for example by introducing a protease-specific oligopeptide in front of a target protein, which is then removed in vitro by the respective protease, for example, factor Xa, enterokinase, and cathepsin C (Belagaje et al. 1997. Protein Sci. 6: 1953-1962).

An object of the invention has thus been to provide an immunogen variant retaining the N-terminal methionine (Met-var24).

Common techniques enabling the evaluation of beta amyloid deposition and effects are imaging techniques with PET and MRI. However, these techniques are very costly, not yet fully validated and acknowledged in clinical routine, require highly qualified staff and specific instruments, are not indicative of a real biological response and therefore are used only in exceptional cases and accompanied by a chemical-physical investigation. Another object of the present invention is to provide an in vitro method for the detection of relevant antibodies against abeta amyloid protein when the subject is immunized with Met-var24 or X-var24 in order to provide a therapeutic guidance and select the correct individuals for continued treatment.

### SUMMARY OF THE INVENTION

The inventors have discovered new abeta immunogen constructs that generates antibodies in AD patients recognizing oligomeric and aggregated forms of abeta. These immunogens are composed of 3 copies of the N-terminal abeta amino acids 1-12 interspersed by the tetanus epitopes P2 and P30 (SEQ ID NO: 1) and an extra N-terminal amino acid such as methionine added to the first copy of abeta (SEQ ID NO: 2 and 3). Indeed, without wishing to be bound by theory, the inventors have discovered that the addition of methionine to the N-terminus of var24 (Met-var24) results in a immunogen with the ability to induce antibodies to oligomeric and aggregated abeta, e.g., the pathogenic forms of abeta, specifically, and not “free N-terminal abeta” reactive antibodies thereby providing a more effective treatment for AD than var24 without methionine. It's further envisaged that the extra N-terminal amino acids in some embodiments could be another amino acid than methionine (called “X” (X-var24) and defined herein below).

An object of the present invention is a method of treating a patient having Alzheimer's disease with an effective amount of Met-var24 or X-var24.

Another aspect of the invention is a method of treating a patient having Alzheimer's disease by treatment with an effective amount of Met-var24 or X-var24, wherein said patient upon earlier treatment with Met-var24 or X-var24 has been shown to raise a beneficial antibody titre response as defined in the present invention.

The present invention is also based on the detection of antibody titre levels in Alzheimer's disease patients that has received one or more immune therapies with Met-var24 or X-var24 by obtaining a plasma sample from the patient and evaluating the titre in an immunoassay or radioimmunoassay of the invention.

The inventors can furthermore quantify the treatment response of Met-var24 or X-var24 treated patients by evaluating titre in an assay, such as an ELISA or MSD. By the method of the present invention the quality of the antibodies raised by active immunization can thus be assessed, thereby enabling an effective monitoring of the therapy and determining when or who will benefit continuation of the immunization or a given dosage regime.

An aspect of the invention is an in vitro method for quantification of antibodies from Alzheimer's disease patients comprising the steps of
a) Taking a plasma sample from a patient,
b) Analysing the plasma sample in an immunoassay of the invention.

Yet a further aspect of the invention relates to the use of Met-var24, X-var24, oligomeric and/or aggregated abeta or a fragment of said abeta in an immunoassay in order to determine the treatment response in patients treated with Met-var24 or X-var24.

In still a further aspect the invention relates to Met-var24, X-var24, oligomeric and/or aggregated abeta or a fragment of said abeta for use in determining the treatment response in Alzheimer's disease patients treated with Met-var24 or X-var24.

### BRIEF DESCRIPTION OF DRAWINGS

FIG. 1 shows an illustrative experiment of the binding of Biosimilar (BS) aducanumab to Var24 and Met-var24 as tested in plates coated with Met-var24. Var24 competed for BS aducanumab binding less efficiently that Met-var24 as indicated by >70-fold increased IC50.

FIG. 2 shows an inhibition assay with BS bapineuzumab in plates coated with abeta 1-28 (the N-terminal amino acids 1-28 of abeta). Inhibition using Var24 and a abeta trimer construct of abeta 1-12 (composed of 3 the N-terminal abeta 1-12 amino acids each linked via an 8-mer of glycine residues) was stronger than by Met-var24.

FIG. 3. shows data from binding analysis of i) AD immune plasma from patients treated with Met-var24 (filled boxes) and ii) BS aducanumab (open boxes) in plates coated with Met-var24. BS aducanumab and AD immune plasma, respectively were incubated 60 min. with increasing concentration of Met-var24 (indicated below by x-axis). The AD immune plasma was pooled samples from patients receiving 4×250 ug doses. Y-axis shows binding as function of increasing concentrations of Met-var24. Data points represent mean of duplicates. Both BS aducanumab and AD immune plasma showed efficient binding of Met-var24 as indicated by sub-nanomolar IC50 values.

FIG. 4: shows an analysis using AD plasma from patients immunized with Met-var24 in an inhibition assay using Met-var24 and an abeta 1-12 trimer (described above in FIG. 2) in plates coated with Met-var24. Binding of AD immune plasma in Met-var24 coated plates is inhibited although to a lesser degree with the abeta 1-12 trimer construct than Met-var24 confirming that N-terminal abeta specificity of Met-var24 induced antibodies.

FIGS. 5A-5D. Met-var24 anti-abeta antibody in Tg2576 Mice. Antibody concentrations were analysed using a capture ELISA based on immobilization of abeta 1-40. FIG. 5A. The kinetics of appearance of anti-abeta antibody in the sera of individual mice which has been given immunizations with Met-var24 formulated in strong adjuvants CFA/IFA or Quil-A. Bleedings were obtained 28 days after each immunization. Data are reported as average values (FIG. 5B). Isotypes of antibodies in the pooled sera of immunized mice. FIG. 5C. A anti-abeta antibody level in the pooled mice sera after 5 to 11 immunizations with Met-var24 formulated in Quil A. FIG. 5D. The IgG isotype distribution of the anti-abeta responses in the pooled sera. Error bars indicate the average±SD of individual animals for FIG. 5A and the pooled sera from 3 different ELISA for FIGS. 5B, 5C, and 5D. Bars represent average±SD for n=20 and n=15 in immunized and control mice, respectively.

FIGS. 6A-6B. Met-var24 Immunizations Reduce Abeta Load

6E10-immunoreactive and diffuse abeta plaques (FIG. 6A) and (FIG. 6B) ThS-positive abeta plaques in the brains of 15- to 17-month old Tg2576 mice immunized at an early age (*** P<0.001, n=20 and n=18 in immunized and control mice, respectively). Representative images of immunized and control mice hemibrains stained with 6E10 and ThS are presented. Magnification 4×, scale bar=200 μm.

FIGS. 7A-7B. Met-var24 Reduces Glial Activation and Astrocytosis without Increasing Cerebral Amyloid Angiopathy

Immunization with Met-var24 reduces glial activation (FIG. 7A) and astrocytosis (FIG. 7B) in the brains of 15- to 17-month-old TG2576 mice. Image analysis of hemibrains stained with anti-MHC II (FIG. 7A) and anti-GFAP (FIG. 7B) antibody showed significantly less microglia activation and astrocytosis, respectively, in immunized mice in comparison with control mice (*** P<0.001). Original magnification 4×, scale bars=200 μm. Boxed brain areas presented in detail using higher original magnification (10×, scale bars=200 μm).

FIGS. 8A-8B. Immunostaining of Plaques and Vascular Abeta in AD Cortex

FIG. 8A: Immunostaining of plaques and vascular abeta using plasma from Met-var24 immunized Jucker (APPPS1-21) mice, alone or preincubated with increasing concentrations of abeta construct using the N-terminal abeta 1-28 amino acids (abeta 1-28 construct) (0-1000 nM). Arrows indicate staining of plaques and arrow heads indicate vascular abeta.

FIG. 8B: Immunostaining of plaques and vascular abeta using plasma from Jucker (APPPS1-21) mice immunized with Met-var24, alone or preincubated with increasing concentrations of Met-var24 (0-1000 nM).

FIG. 9 Binding Characteristics of Met-var24-induced Antibodies

Summary of observed IC50 values of Met-var24 and abeta1-28 constructs obtained in solution competition to antibodies in plasma from immunized Tg2576 mice, Jucker (APPPS1-21) mice and Cynomolgus monkeys. Relative antibody binding to human AD cortical abeta plaques are included.

FIGS. 10A-10D. Effect of Met-var24, P2, and P30 on CD3+CD4+ Proliferation in human PBMCs from healthy donors. Plot showing relation between stimulation index (SI) after in vitro stimulation with Met-var24 and tetanus toxoid (TT) in young (FIG. 10A) and elderly (FIG. 10B). SI after re-stimulation with Met-var24 compared to tetanus peptides P2 and P30 in young donors is shown in FIG. 10C and FIG. 10D. Stimulation index on Y axis ranging 0-40 in FIG. 10A and FIG. 10B and otherwise stimulation indexes are ranging 0-20.

### DETAILED DESCRIPTION OF THE INVENTION

The inventors of the present invention provide for the first time a sensitive and predictive method intended for the detection of oligomeric specific antibodies raised upon active immunization against abeta. The invention also relates to new abeta molecules, Met-var24 and X-var24, which provides promising treatment for AD patients. Without wishing to be bound by theory, the inventors have discovered that the addition of Met to the N-terminus of var24 results in the immunogen gaining the ability to induce antibodies to oligomeric and aggregated forms of abeta, e.g., the pathogenic forms of abeta, specifically, thereby providing a more effective treatment for AD than a var24 construct without methionine. Indeed, as is shown in Example 1, plasma from AD patients immunized with Met-var24 has antibodies specific to oligomeric forms of abeta, do not bind monomeric abeta and has characteristics very close to BS aducanumab. In contrast constructs without N-terminal residues, for example comprising N-terminal abeta residues 1-28 or 1-12, has characteristics closer to BS bapineuzumab and are more directed to monomeric abeta.

Embodiments of the inventions will be described below and in the claims.

I. Var24 Constructs and Formulation

The active immunotherapy with var24 constructs comprises a trimer of abeta 1-12 interspersed by the tetanus toxoid epitopes P30 (bold) and P2 (italic) as shown in SEQ ID NO:1, 2 and 3. An N-terminal amino acid may additionally be present; in SEQ ID NO:2 the N-terminal amino acid is methionine (Met-var24), in SEQ ID NO:3 the N-terminal amino acid may an amino acid as defined below (X-var24).

Var24
(SEQ ID NO: 1)
DAEFRHDSGYEVFNNFTVSFWLRVPKVSASHLEDAEFRHDSGYEVQYIKA
NSKFIGITEL DAEFRHDSGYEV
Met-var24
(SEQ ID NO: 2)
MDAEFRHDSGYEVFNNFTVSFWLRVPKVSASHLEDAEFRHDSGYEVQYIK
ANSKFIGITEL DAEFRHDSGYEV
X-var24
(SEQ ID NO:3)
XDAEFRHDSGYEVFNNFTVSFWLRVPKVSASHLEDAEFRHDSGYEVQYIK
ANSKFIGITEL DAEFRHDSGYEV

X=an amino acid, preferably a natural amino acid and even more preferred a hydrophilic residue, such as Ser (S), Thr (T), Asn (N), and Gln (Q); an aliphatic residue such as Gly (G), Ala (A), Val (V), Leu (L), and Ile (I); or a non-polar residue such as Cys (C) and Pro (P).

Met-var24 and X-var24 may be administered parenterally, by injection, for example, either subcutaneously, intracutaneously, or intramuscularly. Preferably Met-var24 or X-var24 is administered 1, 2, 3 or 4 times or more.

Dosage ranges are of the order of several hundred micrograms active ingredient per immune therapy with a preferred range from about 5 μg to 2,000 μg (even though higher amounts in the 1-10 mg range are contemplated), such as in the range from about 50 μg to 1,000 μg, preferably in the range from 100 μg to 500 μg and especially in the range from about 200 μg to 500 μg. Suitable regimens for initial administration and booster shots are also variable but are typified by an initial administration followed by subsequent inoculations or other administrations.

The immune response of Met-var24 and X-var24 can be enhanced if the immuneconstructs further comprises an adjuvant substance. Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

The application of adjuvants includes use of agents such as aluminium hydroxide or phosphate (alum), commonly used as 0.05 to 0.1 percent solution in buffered saline, admixture with synthetic polymers of sugars (e.g. Carbopol®). Other adjuvants may be AS01 (comprising MPL, liposome and QS21), ISS (comprising oligonucleotides), QS-21 Stimulon® (comprising Saponin), AS02 (comprising MPL, oil-inwater emulsion and QS-21), IC31® (comprising peptides and oligonucleotides), CAF01 (comprises liposomes), dmLT (comprises detoxified proteins), Flagellin (comprises flagellin linked to an antigen), ISCOMATRIX® (comprises ISCOM (Saponins+cholesterol+phospholipids), Matrix-M™ (comprises ISCOM (Saponins+cholesterol+phospholipids), MPL-SE (comprises MPL and Oil-in water emulsion) PCPP (comprises synthetic polyelectrolytes), and PLG (comprise polymeric microparticles).

Injectables can be prepared in conventional forms, either as liquid solutions or suspensions; solid forms suitable for solution or suspension in liquid prior to injection or as emulsions.

Suitable excipients are, for example, water, saline, dextrose, glycerol or ethanol. In addition, if desired, the pharmaceutical compositions to be administered can also contain minor amounts of nontoxic auxiliary substances such as wetting or emulsifying agents, pH buffering agents, stabilizers, solubility enhancers, and other such agents, such as, for example, sodium acetate.

Excipients may include amino acids such as arginine, aspatate, glycine, glutamate, lysine, and proline; antioxidants such as ascorbic acid, EDTA, and malic acid; proteins such as albumin and gelatin; sugars/sugar such as alcohols, sucrose, trehalose, lactose, dextrose, glycerol, sorbitol, and mannitol and surfactants such as Tween.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent or sterile solution just prior to use. The solutions can be either aqueous or non-aqueous.

If administered intravenously, suitable carriers include physiological saline or phosphate buffered saline, and solutions containing thickening and solubilizing agents, such as glucose, polyethylene glycol, and polypropylene glycol and mixtures thereof.

Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p-hydroxybenzoic acid esters, thiomersal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect.

The unit-dose parenteral preparations are packaged in an ampoule, a vial or a syringe with a needle. All preparations for parenteral administration must be sterile, as is known and practiced in the art.

The final pharmaceutical composition of Met-var24 is envisaged to contain substantially pure Met-var24 without any other variants of N-terminal var24 such as var24 or X-var24. In some embodiments 90% or more of the N-terminal methionine variant of var24, such as e.g. 95%, 98%, 99% or even 100% is Met-var24 out of the total amount of N-terminal Var24 variants (such as var24 or X-var24) that may be made during the E. coli process and the following processing and purification leading to a pharmaceutical composition.

II. Patient Treatment and Monitoring

The present invention relates to a method of treating a patient having Alzheimer's disease (AD) by treating said patient with an effective amount of Met-var24 or X-var24. The treatment is according to one embodiment by subcutaneous or intramuscular administration.

According to one embodiment, the invention relates to a sub-population of patients, in particular AD patients, identified by raising a titre above 1.000 when treated with Met-var24 or X-var24. Said titre may be the endpoint titre, that is the highest titre measured after immunization (which maximum differs from subject to subject, but typically occurs within the first approximately 2-3 weeks). The patient may in certain embodiments be a patient that has received an earlier immunization with tetanus toxin or an immunization construct that comprises P2 (QYIKA NSKFIGITEL (SEQ ID NO: 4)) and/or P30 (FNNFTVSFWLRVPKVSASHLE (SEQ ID NO: 5)). Alternatively, the patients may be immunized with tetanus toxin or a immunization construct that comprises P2 and/or P30 before being treated with Met-var24 or X-var24.

The treatment effect of the treatment with Met-var24 or X-var24 is according to an embodiment done by evaluating induced titre or IC50 of a sample from a patient. According to one embodiment the evaluation is made after the patient has been treated 1, 2, 3, 4 or more times with Met-var24 or X-var24.

The evaluation is according to a further embodiment made in an immunoassay comprising the steps of
Applying a sample obtained from a patient in an immunoassay according to the invention under appropriate binding conditions, and measuring the antibody titre or IC50.

The term “sample” refers to a sample of tissue or fluid isolated from a subject, including but not limited to, for example, plasma, serum, spinal fluid, whole blood or any blood fraction. In particular, the sample may be in the form of a blood sample, plasma sample or serum sample obtained from a patient of the invention. Preferably the sample is a plasma sample. According to an embodiment, the sample can be used to determine the titre levels of the antibody response, where the titre is expressed as the inverse of the greatest dilution (in a serial dilution) that still gives a positive result in the assays disclosed herein. ELISA is a common means of determining antibody tires.

In one embodiment, the immune therapy of the patients is continued if the antibody titre and/or IC50 shows that a beneficial antibody response is induced in the immunized patient and is discontinued if the titre and/or IC50 shows a lack of beneficial antibody response in the immunized patient.

According to one embodiment the IC50 of the sample indicative of a beneficial antibody response is in the range of about 200 to about 1000 pM, and thus a IC50 higher is indicative of a none beneficial antibody response. Alternative, the titre itself can be used to define a beneficial response, and it is envisaged that at titre above 1,000, such as above 5.000, such as above 10.000, above 20.000, above 30.000, above 40.000, above 50.000 or above 80.000 indicates a beneficial response, whereas a titre below 1.000 are indicative of none-responding.

The invention also relates to a method of treating a patient having Alzheimer's disease by treatment with an effective amount of Met-var24, wherein said patient upon earlier treatment with Met-var24 has been shown to raise a titre response comprising a beneficial antibody response as disclosed herein-above.

By the term “Alzheimer's Disease” (or abbreviated “AD”) in the present invention is intended to mean a patient with the presence of amyloid and tau in CSF and neurodegeneration changes (as assessed by MR scanning, CSF measurements of neurofilament light, neurogranin and/or tau). Clinical manifestations including mild symptoms of dementia (cognitive and functional deterioration), also including patients with pre-dementia (meaning the patients have cognitive impairment but no significant functional deterioration). It may be envisaged that certain patients will have no measurable sign of cognitive impairments, but presence of pathological reduced amyloid and pathological increased tau in CSF, optionally positive in brain amyloid PET (National Institute Aging NIA-AA Research Framework: Towards a Biological Definition of Alzheimer's Disease, 2017).

According to one embodiment the AD patients of the invention is defined as stage 0, 1, 2 and 3 AD as defined in the recent FDA draft Guidance for Industry: Early AD and including only patients fulfilling the requirements of biomarker positivity for both amyloid and tau according to the recent NIA-AA Research Framework for AD (2018). The population of stage 0, 1, 2 and 3 AD is consistent with preclinical AD (with subjective cognitive complaints) and MCI due to AD as defined in the EMA Guideline on the clinical investigation of medicines for the treatment of Alzheimer's disease.

Biomarker against amyloid abeta, can be a positive amyloid PET scan (using e.g. PET tracer 18^{F}-flutemetamol (Palmquist et al., 2015, Neurology, 1240-1249) and CSF biomarkers may be amyloid-beta 1-42 and total-tau or phosphor-tau (Palmquist et al., 2015, Neurology, 1240-1249; Olsson et al, 2017 Exp. Rev. of Neurot., VOL. 17, NO. 8, 767-775). History of cognitive decline can be assessed using Clinical Dementia Rating (CDR)-Global score of 0.5 (Marcel et al., 2011, Am J of Alzheimer's Disease & Other Dementias, 26(5) 357-365; O'Bryant et al., 2010, Arch Neurol. June; 67(6): 746-749) or Mini-Mental State Examination (MMSE) above 24 (Perneczky et al., 2006, Am J Geriatr Psychiatry 14:2, February).

The patients have according to one embodiment received a dose of 50 μg, 100 μg, 250 μg, 500 μg or 1000 μg Met-var24. According to another embodiment the patients may receive 2 simultaneous dosages of 250 μg Met-var24, for example one dosage of 250 μg Met-var24 in each shoulder. An advantage of immunizing at different locations with higher dosage of Met-var24 is that it may reduce local reactions such as redness, swelling and edema that has been observed in some preclinical studies.

Emphasis was given to design a molecule with the potential to utilize pre-existing tetanus peptides (P2 and P30) specific memory T cell pools to enhance the antibody response in the elderly populations of AD patients. It is well documented that peripheral naïve T-cell populations are reduced with age, whereas the numbers of memory and terminally differentiated T cells increase. Met-var24 or X-var24 displays tetanus-derived peptides P2 and P30, thus designed for efficient response of both naïve and memory T cells. The response to Met-var24 or X-var24 may be augmented in tetanus-vaccinated elderly population. A booster immunization with tetanus, or just P2 or P30 epitopes, prior to immune therapy with e.g. Met-var24 may also enhance the response to Met-var24. By boosting with e.g. P30 naïve T cells may be activated, and this may be particular beneficial in elderly with a lower proportion of expected responders due to senescence of the immune system. Alternatively, people that has already received a tetanus-vaccination may be selected as a target population because their response to e.g. Met-var24 may be augmented.

Met-var24 may be formulated in an appropriate adjuvant and contain further excipients or carriers as disclosed herein.

According to an embodiment the patient may be a Caucasian having AD and the dosage may be 250 μg Met-var24 or above, such as a dosage comprising 2 times 250 μg Met-var24 or a single dosage of 500 μg Met-var24. The patient may in certain embodiments be a patient that has received an earlier vaccination with tetanus toxin or a vaccine that comprises P2 and/or P30. Alternatively, the patients may be vaccinated with tetanus toxin or a vaccine that comprises P2 and/or P30 before being treated with Met-var24.

The treatment of the patients may be done every 2 week, every 4 week or every 6 week. Alternatively the treatment of the patients may be done every ¼ years, every ½ year or for example every year.

In the present context, “treatment” or “treating” is intended to indicate the management and care of a patient for the purpose of alleviating, arresting, partly arresting, removing or delaying progress of the disease. The progress can be the clinical manifestations of the disease or one or more of the patient criteria mentioned herein-above. The patient to be treated is preferably a mammal, in particular a human being.

In the present context, the term “therapeutically effective amount” of a compound means an amount sufficient to alleviate, arrest, partially arrest, remove, reduce one or more symptoms of, or delay the onset or progression of a given disease. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

III. Antibody

In some embodiments, the methods described herein further provide for producing an antibody to oligomerized abeta comprising immunizing an animal (such as a mammal, incl humans) with Met-var24 or X-var24; and isolating an antibody that specifically binds to oligomerized abeta from the animal. Such an antibody can be used to delay onset or progression of or to treat Alzheimer's disease, or to diagnose Alzheimer's disease by measuring the level of oligomerized abeta in a subject.

The term “immunizing” refers to the ability of a substance to cause a humoral and/or cellular response in a subject, whether alone or when linked to a carrier, in the presence or absence of an adjuvant.

The antibody or antigen-binding fragment thereof may be an IgG1, IgG2, IgG3, IgG4, or may have an immunoglobulin constant and/or variable domain of an IgG1, IgG2, IgG3, IgG4. In some embodiments, the antibody is a bispecific or multispecific antibody. In some embodiments, the antibody is a recombinant antibody, a polyclonal antibody, a monoclonal antibody, a humanized antibody or a chimeric antibody, or a mixture of these. In some embodiments, the antibody is a human antibody, e.g., a human monoclonal antibody, polyclonal antibody or a mixture of monoclonal and polyclonal antibodies. Antigen-binding fragments may include a Fab fragment, a F(ab′)2 fragment, and/or a FV fragment CDR3. Antibodies can be raised against Met-var24/X-var24. Antibodies can be generated by injecting an animal, for example a rabbit or goat or mouse, with Met-var24/X-var24. In order to prepare polyclonal antibodies, Met-var24/X-var24 can be synthesized in bacteria by expression of corresponding DNA sequences in a suitable cloning vehicle. Met-var24/X-var24 can then be purified, coupled to a carrier protein and mixed with Freund's adjuvant (to help stimulate the antigenic response by the rabbits) and injected into rabbits or other laboratory animals. Alternatively, the polypeptides can be isolated from cultured cells expressing the protein. Following booster injections at bi-weekly intervals, the rabbits or other laboratory animals are then bled and the sera or plasma isolated. The sera or plasma can be used directly or purified prior to use, e.g., by methods such as affinity chromatography, Protein A-Sepharose, Antigen Sepharose, Anti-mouse-Ig-Sepharose. The sera can then be used to probe protein extracts run on a polyacrylamide gel to identify Met-var24/X-var24. Alternatively, Met-var24/X-var24 can be made and used to inoculate animals. To produce monoclonal Met-var24/X-var24 antibodies, mice are injected multiple times (see above), the mice spleens are removed and re-suspended in a phosphate buffered saline (PBS). The spleen cells serve as a source of lymphocytes, some of which produce antibodies of the appropriate specificity. These are then fused with a permanently growing myeloma partner cell, and the products of the fusion are plated into a number of tissue culture wells in the presence of a selective agent such as HAT. The wells are then screened by ELISA to identify those containing cells expressing useful antibody. These are then freshly plated. After a period of growth, these wells are again screened to identify antibody-producing cells. Several cloning procedures are carried out until over 90% of the wells contain single clones which are positive for antibody production. From this procedure a stable line of clones is established to produce the antibody. A monoclonal antibody can then be purified by affinity chromatography using Protein A Sepharose, ion-exchange chromatography, as well as variations and combinations of these techniques (See e.g., U.S. Pat. No. 6,998,467). For antibodies to be used in therapy in humans, they may be ‘humanized’. Humanization of antibodies involves replacing native mouse sequences with human sequences to lower the chance of an immune response once the therapeutic antibody is introduced into humans. In some embodiments, human antibodies (e.g., identified from libraries of human antibodies) may be used.

IV. Assay

The immunoassay described herein may be in the form of a competitive assay or non-competitive assay (such as a one-site competitive assay or two-site competitive assay) and use labels such as enzymes (e.g. enzyme-linked immunosorbent assays (ELISAs) using horseradish peroxidase (HRP), alkaline phosphatase (AP) or glucose oxidase), radioactive isotopes (e.g. radioimmunoassay) or fluorogenic reporters. Many references are available to provide guidance in applying the above techniques (Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

Meso Scale Diagnostics (MSD) has developed electrochemiluminescent detection technology that can be used as an alternative approach to traditional ELISAs. MSD assays utilize SULFO-TAG labels instead of peroxidase, and microplates with carbon electrodes integrated into the bottom of each well rather than polystyrene microplates. SULFO-TAG labels emit light upon electrochemical stimulation initiated at the electrodes in each well, and signal is measured. The secondary (or detection) antibody can be directly labelled with a SULFO-TAG ester or a SULFO-TAG labelled anti-species antibody e.g. anti-mouse if the detection antibody is raised in mouse and provided that the detection antibody is raised in a different species than the capture (or primary) antibody.

The MSD platform offers significant advantages to detect and quantify protein and protein-complexes in complex matrices. These include a high sensitivity and broad dynamic range allowing measurements of the levels of protein or protein complexes over a broad range of concentrations (typically covering 3.5 to 5 logs of protein concentration) thereby eliminating requirements to re-test samples saving both on time and reagents. Robust and reproducible instrumentation and small sample requirements—typically only 25 μL.

In one embodiment the immunoassay can be in the form of an ELISA or MSD coated with oligomeric and or aggregated abeta, or a fragment of these abeta species, or Met-var24, or X-var24 to which the sample is added and incubated before labelled anti-IgG antibody is finally added. Preferably Met-var24 is used in coating. The readout may be the titre or the calculated IC50 as shown in the Examples.

The invention also relates to an in vitro method for quantification of antibodies from an Alzheimer's disease patient comprising the steps of
c) Taking a sample from a patient,
d) Analyzing the sample in an immunoassay.

The immunoassay comprises according to an embodiment a first step of absorbing/adsorbing oligomeric and/or aggregated abeta, or a fragment of said abeta species, or Met-var24 or X-var-24, to a solid phase, such as a plate, in the immunoassay. Preferably Met-var24 is used in the first step of absorbing/adsorbing to the solid phase.

The sample is taken from a patient that has been treated with Met-var24 1, 2, 3, 4 times or more according to an embodiment of the invention.

The data can be used to calculate the IC50 and an IC50 in the range of about 200 to about 1000 pM indicates a positive titre against the patients abeta in the brain. Alternative, the titre itself can be used to define a beneficial response, and it is envisaged that at titre above 1.000, such as above 5.000, above 10.000, above 20.000, above 30.000, above 40.000, above 50.000 or above 80.000 indicates a beneficial response, whereas a titre below 1.000 are indicative of none-responding.

By the term “titre” in the present invention is intended endpoint titre defined as a dilution factor where the specific signal in plasma sample from an individual treated with Met-var24 or X-var24 is reduced to a level of average background+3× standard deviation (sometimes indicated as cut-line with stippled lines in graphs). Specific signal is defined as signal for antibody of interest in individual plasma sample from individual receiving treatment subtracted signal in plasma from same patient before treatment (pre-immune level).

Also provided herein are in vitro method for quantification of oligomerized abeta from a patient. Such an assay can be used to diagnose or to prognosis of Alzheimer's disease.

The method comprises the steps of taking a sample from a patient, applying the sample to an immunoassay comprising oligomerized abeta described herein (e.g. Met-var24) under appropriate binding conditions; and measuring the level of binding of antibodies in the patient sample to the oligomerized abeta (e.g. Met-var24).

In some embodiments, in any of the assays described herein, the level of binding (e.g., the level of antibodies or oligomerized abeta measured) can be compared to a control. In some embodiments, the control is a sample from a subject who does not have Alzheimer's disease. In some embodiments, the control is the average level from a population of subjects. In some embodiments, the population of subjects do not have Alzheimer's disease. In some embodiments, the control is a sample from the patient at an earlier time point (for example the same patient that has not received treatment of Met-var24, or the same patient undergoing treatment with Met-var24).

In a specific embodiment, the in vitro assay of the invention comprises 1, 2, 3, 4, 5 or all the following steps
a. Coating of the plate (ELISA or MSD plate) with Met-var24,
b. Optionally, blocking the plate,
c. Optionally, washing the plate 1, 2, 3, 4 or 5 times,
d. Adding Met-var24 pre-incubated with a sample from a patient (e.g. a plasma sample as described herein above) that has received Met-var24 treatment, so as to generate a series of patient samples with Met-var24 in different concentrations,
e. Optionally, washing the plate 1, 2, 3, 4 or 5 times,
f. Adding a sulfo-tagged anti-human IgG,
g. Incubating the plate between about 30 minutes to about 80 minutes,
h. Optionally, washing the plate 1, 2, 3, 4 or 5 times
i. Optionally, adding a reading buffer
j. Obtaining the results, e.g. from a plate reader such as a MSD plate reader

V. Kit

The invention also relates to a kit comprising the immunoassay of the invention and, according to an embodiment, also a medicament for treating Alzheimer's disease, such as Met-var24 or an anti-abeta antibody like aducanumab.

According to one embodiment, the Kit comprises an immunoassay with oligomeric and/or aggregated abeta, or a fragment said abeta, or Var24, Met-var24 or X-var-24. Preferably Met-var24 is used.

The immunoassay is an ELISA or MSD according to one embodiment.

In one embodiment, the kit can be used to monitor the treatment response of AD patients by measuring the titre or IC50 of Met-var24 treated patients.

According to one embodiment the patient has been treated 1, 2, 3, 4 or more times with Met-var24.

According to one embodiment the IC50 indicative of a beneficial antibody response is in the range of about 200 to about 1000 pM or above, and thus a titre higher is indicative of a none beneficial antibody response. Alternative, the titre itself can be used to define a beneficial response, and it is envisaged that at titre above 1.000, such as above 5.000, above 10.000, above 20.000, above 30.000, above 40.000, above 50.000 or above 80.000 indicates a beneficial response, whereas a titre below 1.000 are indicative of none-responding.

The term “oligomeric abeta” in the present invention is intended to define a form of abeta wherein at least two, such as e.g. 3, 4, 5 or more, abeta 1-39 to 1-43 molecules are associated.

The term “aggregated abeta” in the present invention is intended to define aggregated abeta 1-39 to 1-43 molecules which can be isolated from a synthetic or biological sample using low centrifugation force about 10.000 g, for example at 16,000 g for 30 min (Mok and Hawlet, 2006, Methods in Enzymology, Vol. 413, 199-217). Synthetic variants of these are also envisaged.

By the term “abeta fragment” in the present invention is intended refer to peptide of abeta 1-43 molecules with a size larger than 4 amino acids.

By the term “appropriate binding conditions” as used in the present invention is intended to mean antibody-antigen interaction at conditions that reflect physiological condition e.g. Phosphate buffered solution pH 7.4 with 1 mg/ml BSA or 20 mM Tris, 150 mM NaCl, 1 mg/ml BSA.

### Further Embodiments of the Invention

### Method of Treatment (Embodiment “E”)

E1. (Embodiment 1). A method for treatment of a patient in the need thereof with an effective amount of Met-var24 or X-var24, such as a patient with Alzheimer's Disease, e.g. in a Caucasian population.

E2. The method according to E1 wherein the patient is treated with Met-var24.

E3. The method according to E1-E2, wherein an evaluation of the treatment response of the treatment is done by evaluating the induced end point or maximal titre or IC50.

E4. The method according to E3, wherein an evaluation of the treatment response is made after the patient has been treated 1, 2, 3, 4 or more times.

E5. The method according to E4, wherein said evaluation is made in an immunoassay comprising the steps of
a. Applying a sample obtained from a patient according to E1 or E2 in an immunoassay coated with oligomeric and/or aggregated abeta, or fragments of said abeta, X-var24 or Met-var24 under appropriate binding conditions,
b. Measuring the antibody tire or IC50.

E6. The method according to E3-E5, wherein immune therapy of the patients is continued if the titre and/or IC50 shows that a beneficial antibody response is obtained in the immunized patient.

E7. The method according to E3-E5, wherein immune therapy of the patients is discontinued if the titre and/or IC50 shows that a beneficial antibody response is not obtained in the immunized patient.

E8. The method according to E 4 or E5, wherein the immunoassay is an ELISA or MSD assay.

E9. The method according to E 4 or E5, wherein a titre above 1.000 indicates a beneficial antibody response.

E10. The method according to E 4 or E5, wherein an IC50 of in the range of about 200 to about 1000 pM indicates a beneficial antibody response.

E11. The method according to anyone of the previous Embodiments, wherein the patient receives a dose of 50 μg, 100 μg, 250 μg, 500 μg or 1000 μg Met-var24, or receives a dosage regime of 2 simultaneous dosages of 250 μg Met-var24. The method may include an embodiment wherein the patient has received a tetanus toxoid vaccine prior to receiving said effective amount of Met-var24, or wherein the patient has received vaccine (or immunization construct) comprising P2 and/or P30 from tetanus toxoid prior to receiving said effective amount of Met-var24.

E12. The method according to anyone of the previous Embodiments, wherein treatment is done every 2 week, every 4 week or every 6 week. Alternatively the treatment may be given approximately every ¼ year, every ½ year or for example yearly.

E12′. A method of treating a patient having Alzheimer's disease by treatment with an effective amount of Met-var24 or X-var24, wherein said patient upon earlier treatment with Met-var24 or X-var24 has been shown to raise a titre response comprising antibodies according to E 1-10, the Alzheimer's disease patient may be a Caucasian

E13 Met-var24 or X-var24 for the use in the treatment of a patient such as a patient with Alzheimer's Disease, e.g. in a Caucasian population.

E13′ Met-var24 or x-var24 for the use according to E13, wherein the use is further as defined in E2-E12.

E14 Met-var 24 for the manufacturing of a medicament.

E14′ Met-var24 according to E14, wherein the use is further as defined in E1-E12.

E15. Met-var24 or X-var24 for the manufacturing of a medicament for use in a method of treating a patient having Alzheimer's disease, e.g. a Caucasian AD patient.

E16. Met-var24 or X-var24 according to E15, wherein the treatment is done by subcutaneous or intramuscular administration.

E17. Met-var24 or X-var24 according to E15, wherein an evaluation of the treatment response of the treatment is done by evaluating the induced titre or IC50.

E18. Met-var24 or X-var24 according to E17, wherein an evaluation of the treatment response is made after the patient has been treated 1, 2, 3, 4 or more times.

E19. Met-var24 or X-var24 according to E17, wherein said evaluation is made in an immunoassay comprising the steps of
Applying a sample obtained from a patient according to E15 in an immunoassay coated with oligomeric and/or aggregated abeta, or fragments of said abeta, X-var24 or Met-var24 under appropriate binding conditions,
Measuring the antibody titre or IC50.

E20. Met-var24 or X-var24 according to E17-E19, wherein immune therapy of the patients is continued if the titre and/or IC50 shows that a beneficial antibody response is obtained in the immunized patient.

E21. Met-var24 or X-var24 according to E17-E19, wherein immune therapy of the patients is discontinued if the titre and/or IC50 shows that a beneficial antibody response is not obtained in the immunized patient.

E22. Met-var24 or X-var24 according to E17-E21, wherein the immunoassay is an ELISA or MSD assay.

E23. Met-var24 or X-var24 according to E17-E22, wherein a titre above 1.000 indicates a beneficial antibody response.

E24. Met-var24 or X-var24 according to E17-E21, wherein an IC50 of in the range of about 200 to about 1000 pM indicates a beneficial antibody response.

E25 Met-var24 or X-var24 according to E15-E24, wherein the patient receives a dose of 50 μg, 100 μg, 250 μg, 500 μg or 1000 μg Met-var24, or receives a dosage regime of 2 simultaneous dosages of 250 μg Met-var24.

E27. Met-var24 or X-var24 according to E15-E26, wherein treatment is done every 2 week, every 4 week or every 6 week.

E28 Met-var24 or X-var24 for the manufacturing of a medicament for use in a method of treating a patient having Alzheimer's disease (e.g. a Caucasian AD patient) by treatment with an effective amount of Met-var24, wherein said patient upon earlier treatment with Met-var24 has been shown to raise a titre response comprising antibodies according to E15-E23.

E29 Met-var24 or X-var24 for the manufacturing of a medicament for use in a method of treating a patient according to E28 wherein the patient has received a tetanus toxoid vaccine prior to receiving said effective amount of Met-var24, or wherein the patient has received vaccine comprising P2 and/or P30 from tetanus toxoid prior to receiving said effective amount of Met-var24.

E30. A method for inducing an immune response to oligomerized abeta in a patient comprising administering an effective amount of Met-var24 or X-var24.

E31. A method for delaying the progression of Alzheimer's disease in a patient comprising administering an effective amount of Met-var24 or X-var24.

### Composition (Embodiments “F”)

F1. A polypeptide comprising the sequence of SEQ ID NO: 2.

F2. A vaccine construct comprising the immunogenically effective amount of the polypeptide of F1 and an adjuvant, wherein the immunogenically effective amount of the polypeptide induces production of antibodies against oligomerized abeta.

F3. A nucleic acid encoding the immunogenic polypeptide of F1.

### Assay Method (Embodiments “B”)

B1. An in vitro method for quantification of antibodies from a patient comprising the steps of
a) Taking a sample from a patient,
b) Analyzing the sample in an immunoassay.

B2. The method according to B1, wherein the patients has or is suspected to have AD.

B3. The method according to B1, wherein the immunoassay comprises a first step of absorbing/adsorb oligomeric and/or aggregated abeta, or a fragment of said abeta, Met-var24 or X-var24 to a solid phase, such as a plate, in the immunoassay. Preferably Met-24 is used.

B4. The method according to B1 and B3, wherein the immunoassay is an ELISA or MSD.

B5. The method according to B1, wherein the sample is taken from a patient that has been treated with Met-var24 1, 2, 3, 4 times or more.

B6. The method according to B1-B5, wherein the data obtained is used to calculate the IC50.

B7 The method according to B6, wherein an IC50 of the titre in the range of about 200 to about 1000 pM indicates a positive titre against the patients abeta in the brain.

B8 The method according to B1-B5, wherein the titre is determined in the sample.

B9 The method according to B8, wherein a titre above 1.000 indicates a beneficial antibody response against the patients abeta in the brain.

B10. A kit for measuring titre or IC50 in a sample.

B11. The kit according to B11, wherein the is for use in diagnosing AD.

B12. The kit according to B11, comprising oligomeric and/or aggregated abeta, or a fragment of said abeta, Met-var24 or X-var24. Preferably Met-24 is used.

B13. The kit according to B10, wherein the immunoassay is an ELISA or MSD.

### Antibodies (Embodiments “A”)

A1. A method of producing an antibody to oligomerized abeta comprising:
immunizing an animal with Met-var24; and
isolating an antibody that specifically binds to oligomerized abeta from the animal.

A2. The method according to A1, wherein the antibody is a polyclonal antibody isolated from the serum of the immunized animal, such as a mammal (incl human).

A3. The method according to A1, wherein the antibody is a monoclonal antibody and the antibody is isolated by obtaining a cell from the animal and generating a hybridoma.

A4. The method according to A3, wherein the hybridoma is generated by obtaining a spleen cell from the immunized animal and fusing the spleen cell to a myeloma cell.

A5. An antibody or antibody fragment prepared according to A1-A4.

A6. The antibody or antibody fragment of A5, wherein the antibody is monoclonal or polyclonal.

A7. The antibody or antibody fragment of A5, wherein the antibody is a full-length antibody.

A8. The antibody or antibody fragment of A5, wherein the antibody fragment is selected from the group consisting of: an Fv fragment (e.g. single chain Fv and disulphide-bonded Fv); a Fab like fragment such as Fab fragment, Fab′ fragment and F(ab)₂ fragment; and a domain antibody such as a single V_{H} variable domain or V_{L} variable domain.

A9. The antibody or antibody fragment of A5-A8, wherein the antibody is selected from the group consisting of antibodies of subtype IgG1, IgG2, IgG3 or IgG4.

A10. The antibody or antibody fragment of A5-A9, wherein the antibody is human or humanized.

A11. A method of delaying the onset or progression of or treating a patient having Alzheimer's disease (AD) by treating said patient with an effective amount of the antibody of any of A1-A10.

A12. The method of A11, wherein the treatment is done by subcutaneous or intramuscular administration.

A13. The method of A11 or A12, wherein wherein treatment is done every 2 week, every 4 week, every 6 week, every 7 week, or every 8 week. Alternatively the treatment may be given approximately every ¼ year, every ½ year or for example yearly

A11. Use of the antibody of any of A1-A10 in delaying the onset or progression of or treating patient having Alzheimer's disease (AD) by administering an effective amount of the antibody of any of A1-A10.

A12. The use of A11, wherein the treatment is done by subcutaneous or intramuscular administration.

A13. The use of A11 or A12, wherein wherein treatment is done every 2 week, every 4 week, every 6 week, every 7 week, or every 8 week.

A14. An in vitro method for quantification of oligomerized abeta from a patient comprising the steps of
a) Taking a sample from a patient,
b) applying the sample to an immunoassay comprising the antibody of any of A1-A10 under appropriate binding conditions; and
c) measuring the level of binding of oligerimized abeta to the antibody.

A15. The method according to A14, wherein the patients has AD.

A16. The method according to A14 or A15, wherein the immunoassay is an ELISA.

A17. The method according to A14-A16, further comprising treating the patient with the antibody.

### Example 1

11 AD patients were treated at time 0, 4, 12 and 24 weeks with 250 μg Met-var24. Plasma samples were collected 4 weeks after dosing and analysed for antibody binding (titre) using Met-var24 coated MSD plates. Plasma from positive responders were pooled and diluted 10,000 fold where after it was incubated with increasing amounts of Met-var24 or 1-12 abeta trimer construct (composed of the 3 repeats of N-terminal abeta 1-12 amino acid each separated by a 8-mer of glycine residues) in solution to compete for binding to the immobilized Met-var24 in order to validate specificity and measure in solution binding of the anti-abeta antibodies in the pooled AD plasma samples to Met-var24 and the abeta 1-12 trimer. BS aducanumab (4 ng/ml) was analysed in parallel. BS aducanumab and pooled plasma samples from AD patients were incubated with increasing concentration of abeta constructs (such as Met-var24, Var24, abeta 1-12 trimer or the N-terminal abeta 1-28 residues) peptides for 60 minutes at 22° C. and subsequently analysed for free antibody in MSD plates coated with 100 ng/ml Met-var24. Plate bound antibody was detected using Sulfo tagged anti-human IgG (1:1500, MSD catalogue # R32AJ). Pooled AD plasma binding to Met-var24 appear efficient and comparable with BS aducanumab binding to Met-var24 due to same range of IC50 value (Table I). Table I shows representative values from multiple testing

TABLE I

| | abeta 1-12 | abeta 1-28 | | |
|---|---|---|---|---|
| mAb IC50 | trimer | residues | Var24 | Met-var24 |
| BS aducanumab | No | No | No | 0.1-0.3 nM |
| | binding | binding | binding | |
| AD plasma | No | — | — | 0.3-0.6 nM |
| | binding | | | |
| BS bapinezumab | 19 nM | 30 nM | 46 nM | No |
| | | | | binding |

# No binding is given when the affinity is 1 μM or below

The strong binding of Met-var24 to BS aducanumab indicates that Met-var24 display abeta epitopes (amino acids 3-9) efficient to aducanumab and thus indicate that Met-var24 epitopes mimics the epitope present a natural oligomeric and aggregated abeta formed in the course of AD.

In conclusion, the plasma from Met-var24 immunization of AD patients induce antibody that bind Met-var24 efficiently and with a binding profile compatible with BS aducanumab.

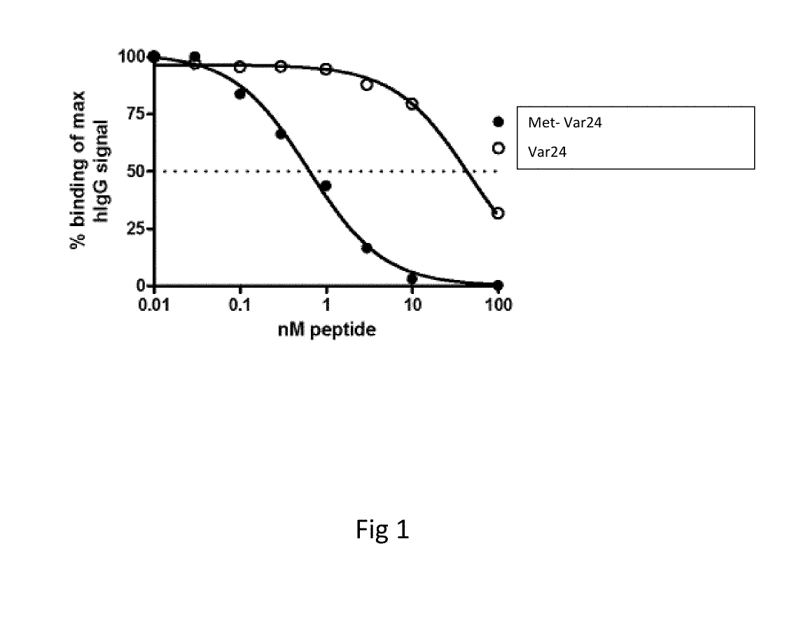

FIG. 1 shows an illustrative experiment of the binding of BS aducanumab to Var24 and Met-var24 as tested in plates coated with Met-var24. Var24 competed for BS aducanumab binding less efficiently that Met-var24 as indicated by >70-fold increased IC50.

Bapineuzumab bind mono, oligomer and aggregated forms of abeta with high affinity and specific for abeta 1-5, and have specificity for the N-terminal Asp (D) of abeta. Bapineuzumab did not improve clinical outcomes in patients with Alzheimer's disease, despite treatment differences in biomarkers observed in APOE c4 carriers. Bapineuzumab failed to meet primary endpoints in phase 3 (Salloway et al, N Engl J Med 2014; 370:322-33).

Met-var24 as well as abeta 1-28 were analysed for binding to biosimilar antibodies of aducanumab (BS Aducanumab) and bapineuzumab (BS Bapineuzumab) using MSD plates coated with Met-var24 and N-terminal amino acids 1-28 of abeta (abeta 1-28), respectively. MSD plates were coated with 100 ng/ml Met-var24 and 500 ng/ml abeta 1-28, respectively (pH 11, boric acid/sodium hydroxide/potassium chloride, FLUKA 33650-1L-R). At these conditions, BS aducanumab but not BS bapineuzumab binds efficiently in MSD plates coated with Met-var24. In contrast BS bapineuzumab but not BS aducanumab binds efficiently in plates coated with abeta 1-28 (Table I).

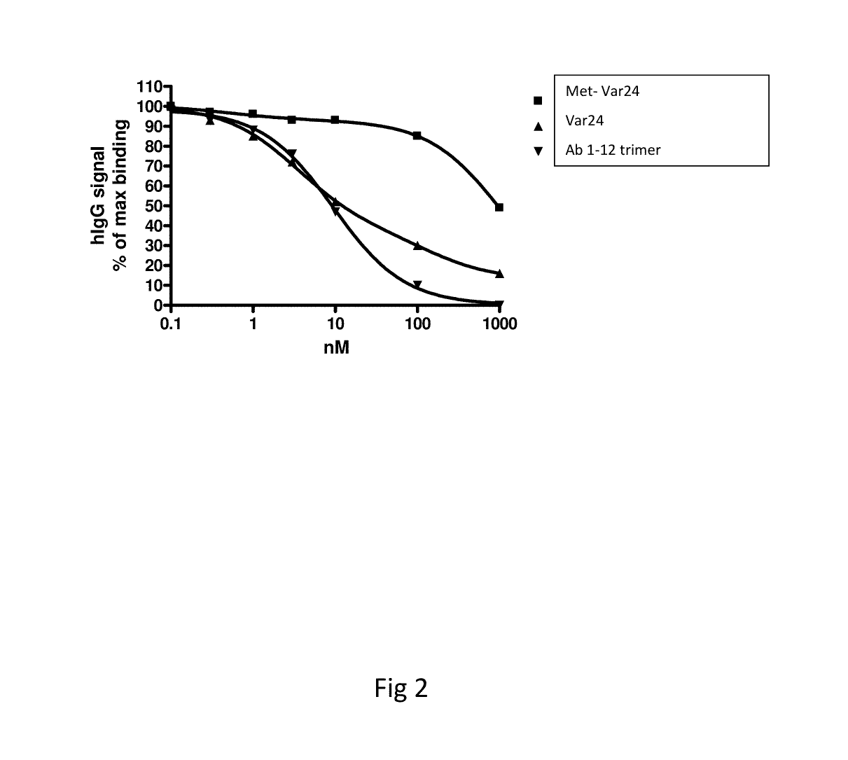

BS bapineuzumab fluid phase binding to Met-var24 was tested in plates coated with abeta 1-28 (inhibition assay). Binding to abeta 1-28 in plates was inhibited weakly by Met-var24 as indicated by an IC50 of 500 nM. In comparison BS bapineuzumab showed binding to Var24 and abeta 1-12 trimer (a trimer of abeta N-terminal 1-12 amino acids each separated with 8-mer flexible linkers using Glycine) with IC50 values about 10-40 nM at these conditions (Table I and FIG. 2).

Met-var24 binding to bapineuzumab show high IC50 value consistent with the blockage of the free N-terminal abeta (Asp-1) required for high affinity interaction of bapineuzumab and abeta. Var24 as well as abeta 1-12 (described above) both with free N-terminal Asp-1, showed in contrast potent binding as indicated by IC50 values about 10 nM

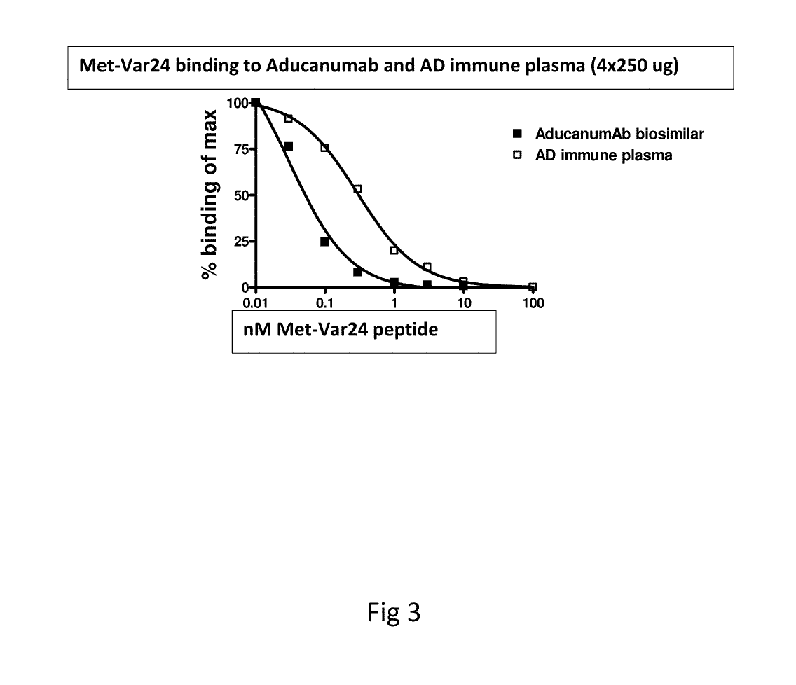

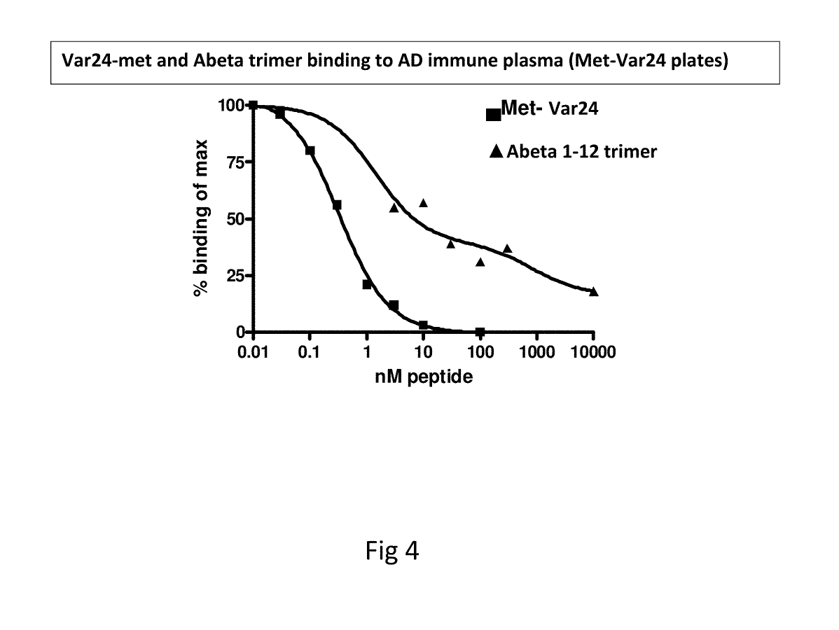

In FIG. 3 data from one of the made binding analysis of AD immune plasma (filled boxes) and BS aducanumab (open boxes) binding to Met-var24 coated plates. BS aducanumab and AD immune plasma, respectively were incubated 60 min. with increasing concentration of Met-var24 (indicated below by x-axis). The AD immune plasma was pooled samples from patients receiving 4 doses of 250 μg. Y-axis shows binding as function of increasing concentrations of Met-var24. Data points represent mean of duplicates. Both BS aducanumab and AD immune plasma showed efficient binding of Met-var24 as indicated by sub-nanomolar IC50 values.

Thus, overall this supports that Met-var24 induces antibodies recognizing oligomeric/aggregated, but not monomeric, abeta with a similar binding potency as aducanumab.

The assay of the invention using Met-var24 is also exceptional good at detecting clinical relevant antibodies against oligomeric/aggregated abeta, like aducanumab. A titre analyses of pooled AD immune plasma after 3 immunizations with 50 μg and 250 μg doses were performed using plates coated with respectively Met-var24 and abeta 1-12 trimer peptides (described above-herein). Pooled plasma samples were serial diluted 300-1,000,000-fold and incubated with the Met-var24 and abeta 1-12 trimer peptides. Bound antibody was detected by sulpho-tagged anti-human IgG (MSD). In plates coated with abeta 1-12-trimer end point titre was only detected in patients receiving 3×250 μg Met-var24 (at a titre about 1:10,000) whereas no antibodies could be detected in plasma from patients dosed with 50 μg. In contrast, when using Met-var24 coated plates titre were measured in patient receiving 50 μg (titre 1:3,000) as well as 250 μg (1:30,000). Immunogenicity of Met-var24 was consequently analysed better using Met-var24 coated plates compared to conventional coated plates.

Heavy Chain (HC) of BS aducanumab
(SEQ ID NO: 6)
QVQLVESGGGVVQPGRSLRLSCAASGFAFSSYGMHWVRQAPGKGLEWVAV
IWFDGTKKYYTDSVKGRFTISRDNSKNTLYLQMNTLRAEDTAVYYCARDR
GIGARRGPYYMDVWGKGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPG
Light Chain (LC) of BS aducanumab
(SEQ ID NO: 7)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Light Chain (LC) of BS bapinuzemab
(SEQ ID NO: 9)
DVVMTQSPLSLPVTLGQPASISCKSSQSLLDSDGKTYLNWLQQRPGQSPR
RLIYLVSKLDSGVPDRFSGSGSGTDFTLKISRVEAEDVGVYYCWQGTHFP
RTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Heavy Chain (HC) of BS bapineuzumab
(SEQ ID NO: 8)
EVQLVESGGGLVQPGGSLRLSCAGSGFTFSNYGMSWVRQAPGKGLEWVAS
IRSGGGRTYYSDNVKGRFTISRENAKNSLYLQMNSLRAEDTAVYYCVRYD
HYSGSSDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG

### Example 2

### Assay Setup A

This assay is an illustration of a method of the MSD assay of the invention using Met-Var24 coating. The method can be for example be used as a “Potency Assay” to identify a possible induction of aducanumab-like antibodies.

### Equipment

Equivalent equipment may be substituted for those listed in the below table.

| Equipment | Manufacturer/Catalogue number |
|---|---|
| MSD Sector S600 | MSD |
| 96-well plates | MSD L15XA |
| Waterbath/Heating Block | Grant TC120, Techne or equivalent |
| Pipettes | Gilson, Rainin, Fisher, Eppendorf, Biohit |
| | Pipettes, sizes as appropriate |

### Reagents

All commercially available reagents are stored, prepared, and used according to the manufacturer's instructions. If required, the preparation of buffers and reagents from stock solutions are listed in this section. Volumes may be adjusted according to need as long as the ratios are kept constant. Equivalent reagents may be substituted for those listed in the below table

| Material Name | Manufacturer/Catalogue Number |
|---|---|
| Carbonate Bicarbonate buffer | Sigma C3041 |
| NP40 | Sigma NP40S |
| PBS | Sigma P5493 |
| BSA fraction V | Roche 1073509400, Sigma A3059 |
| Sulfo tagged anti-human IgG | MSD R32AJ-1 |
| Read Buffer | MSD |

Binding Buffer: PBS pH 7.4 with 0.1% BSA (fraction V)

Prepare binding buffer as follows:

Add 1 g BSA to 900 ml PBS. Adjust pH if necessary to pH7.4. QS volume to 1 litre.

Store at 2-8° C. for 1 month.

Coating buffer: Carbonate buffer pH 9.5±0.3

Prepare carbonate bicarbonate buffer as follows:

Empty the contents of one capsule in 50 mL of deionized water and dissolve. Adjust the pH if necessary. QS volume to 100 ml.

Store at 2-8° C. for up to a 1 month

Blocking buffer: 3% BSA (fraction V), 0.1% NP40 in PBS pH7.4

Prepare blocking buffer as follows:

Add 30 g BSA to 900 ml PBS. Add 1.42 ml of NP40 (NP40 is a 70% solution in water). Adjust pH if necessary to pH7.4. QS volume to 1 litre.

Store at 2-8° C. for up to a 1 month

Washing buffer: 0.1% BSA (fraction V), 0.1% NP40 in PBS, pH 7.4

Prepare washing buffer as follows:

Add 1 g BSA to 900 ml PBS. Add 1.42 ml of NP40 (NP40 is a 70% solution in water). Adjust pH if necessary to pH7.4. QS volume to 1 litre.

Store at 2-8° C. for up to a 1 month

### Procedure

### 1.1. Plate Coating—Day 1

1. A coating buffer containing Met-var24 must be prepared immediately before coating plate.
2. Add 25 μL of the coating buffer per well into all wells needed.
3. Cover the plate with a plate sealer and incubate at 2-8° C. for 16 hours. Alternatively, plates can be coated at room temperature (RT) for 2-3 hours (hr).

### 1.2. Assay Preparation—Day 2

1. Remove the coated plate from 2-8° C. storage and allow to equilibrate at room temperature for at least 30 minutes before use.
2. Remove binding buffer from 2-8° C. storage. Aliquot the volume of binding buffer required and return the stock bottle to 2-8° C. storage.
3. Remove the bottle of wash buffer from 2-8° C. storage. Aliquot the required amount and allow to warm to room temperature for at least 30 minutes. Return the stock bottle to the refrigerator.
4. Decant the coating solution and remove residual buffer by tapping the plate upside down, using clean toweling.
5. Wash the plate 3 times with wash buffer using 150 μL/well for each wash.
6. Remove residual buffer by tapping the plate upside down, using clean toweling.

### 1.3. Blocking—Day 2

1. Block the wells by adding 150 μL of blocking buffer to each well.
2. Cover the plate with a plate sealer and leave at room temperature for 60±10 minutes.
3. After blocking is completed, remove residual buffer by tapping the plate upside down using clean toweling.
4. Wash the plate 3 times with wash buffer using 150 μL/well for each wash. Remove residual buffer by tapping the plate upside down using clean toweling.
   1.4. Preincubation of Met-Var24 Samples with Antibodies
1. Pre-incubate Biosample (e.g. sample from a patient or for example BS Aducanumab or BS Bapineuzumab) in presence of graded concentrations of Met-var24 (Peptide range: 0.1, 1, 10, 100 and 1000 nM) at room temperature for 60 minutes.
2. Add 25 μL/well of sample preparations in duplicate.
3. Cover the plate with a plate sealer and incubate at room temperature for 60 minutes±10 minutes.

### 1.5. Addition of Sulfotag Antibody

1. Approximately five minutes before the end of the incubation, prepare the Sulfotag antibody to dilution of 1:1500 using binding buffer as a diluent.
2. After the end of the sample incubation, remove residual sample by tapping the plate upside down using clean toweling.
3. Wash the plate 3 times with wash buffer using 150 μL/well for each wash. Remove residual buffer by tapping the plate upside down using clean toweling.
4. Add 25 μL of the working Sulfotag solution per well into all wells needed.
5. Cover the plate with a plate sealer and incubate at room temperature for 60 minutes±10 minutes.

### 1.6. Plate Development and Reading

1. After the Sulfotag incubation, remove the solution from the wells.
2. Wash the plate 3 times with wash buffer using 150 μL/well for each wash. Remove residual buffer by tapping the plate upside down using clean toweling.
3. Add 150 μL of the Read buffer per well into all wells needed.
4. Place the plate in the MSD sector 600 reader's drawer, using the proper orientation.
5. Read the plate according to manufacturer's instructions.

### Assay Setup B

According to another embodiment of the invention the Assay may be set up as disclosed below.

### Chemicals, Capturing and Detection Materials

| Material | Manufacturer |
|---|---|
| Goat Anti-Human Antibody | MSD |
| Sulfo-Tag | |
| MSD Sector Imager plate | MSD |
| Carbonate-Bicarbonate | Sigma |
| Buffer Packs | |
| Blocker A kit | MSD |
| Blocker A | MSD |
| Phosphate Buffer | MSD |
| HiSpec assay diluent | Bio-Rad |
| MSD Read buffer (4x) | MSD |
| Milli-Q Water/Ultrapure | |
| Water | |

| Consumable | Supplier |
|---|---|
| Low adhesion plate | NUNC |
| 0.6 mL microtube | Axygen |
| Tubes | Sarstedt/VWR |

### Preparation of Reagents

| Reagent ID | Preparation |
|---|---|
| Coating buffer | Add 1 pouch carbonate-bicarbonate buffer |
| | pack to 500 mL Milli-Q water. Mix by stirring. |
| Blocker A | Weigh out 25.0 g of Blocker A dry powder. |
| | Add 400 mL of Milli-Q water. |
| | Stir until all protein is resuspended. |
| | Add 100 mL of MSD Phosphate buffer (5x). |
| | Vacuum filter the prepared MSD Blocker A |
| | Solution with a 0.2 μM filter into a clean |
| | storage container. |

The volumes used in the preparation of reagents are sufficient for 1 plate (except for buffers).

| Reagent ID | Preparation |
|---|---|
| Wash Buffer | Completely dissolve 1 PBS/Tween Tablet in 1.00 L |
| (PBS/0.05% Tween) | Milli-Q water. |
| Coating Solution | Add 10.0 μL of the AF20513 antibody (17-1410; |
| (150 ng/mL) | 2.00 mg/mL) to 990 μL coating buffer (pre- |
| | dilution). Mix by vortexing. |
| | Take 6.00 mL coating buffer. Take out 45.0 μL |
| | coating buffer and add 45.0 μL pre-dilution to the |
| | coating buffer. |
| | Mix by vortexing. |
| | Prepare within 15 minutes before use. |
| Detection solution | Add 10.0 μL Goat Anti-Human Antibody Sulfo-Tag |
| (0.200 μg/mL) | (17-1422; 500 μg/mL) to 90.0 μL HiSpec assay |
| | diluent (pre-dilution). Mix by vortexing. |
| | Take 6.00 mL HiSpec assay diluent. Take out 24.0 |
| | μL HiSpec assay diluent and add 24.0 μL pre- |
| | dilution to the HiSpec assay diluent. Mix by |
| | vortexing. |
| | Prepare within 15 minutes before use. |
| Read Buffer T | Mix 9.00 mL Read Buffer T (4x) with 9.00 mL |
| (1x) | Milli-Q water. |
| | Prepare within 15 minutes before use. |

### ECLIA System

| | |
|---|---|
| Reader | MSD Quickplex SQ 120 |

### Washer

| Washer | Biotek ELx405 |
|---|---|
| Settings | Dispense flow rate: 5 |
| | Aspirate rate: 3 |

### Procedure

| | | |
|---|---|---|
| | | Pipette |
| Step | Description | ID |

Bring all reagents and materials to room temperature before use, except if specified

Day 1

| | | |
|---|---|---|
| □ 01 | Prepare the capturing solution (150 ng/mL). Prepare within 15 | 1 |
| | minutes before use. | |
| | Pre-dilution (100x): | 2 |
| | Add 10.0 μL (1) of Lu AF20513 (PRActic ID 17-1410; 2.00 | 3 |
| | mg/mL) to 990 μL (2) coating buffer and mix by vortexing. | |
| | Capturing solution (150 ng/mL): | 4 |
| | Take 6.00 mL (3) coating buffer | |
| | Take out 45.0 μL (4) coating buffer | |
| | Add 45.0 μL (4) of the pre-dilution to the coating buffer and | |
| | mix by inverting. | |
| □ 02 | Add 50.0 μL of coating solution to all wells of the MSD plate. | |
| □ 03 | Cover the plate and incubate overnight at nomrnal +4° C. | |

Day 2

| | | |
|---|---|---|
| □ 04 | Prepare the Wash buffer. | |
| | Dissolve 1 PBS/Tween Tablets in 1.00 L Milli-Q water. | |
| □ 05 | Thaw the frozen study samples at room temperature and | |
| | homogenize. | |
| □ 06 | For titration; prepare a 2-fold serial dilution of samples (25.0 μl | |
| | sample + 25.0 μl HiSpec assay diluent) until negative scoring is | |
| | achieved. | |
| □ 07 | Wash the MSD plate 5 times with wash buffer (300 μL/well) and | |
| | tap the plate on a tissue. | |
| □ 08 | Add 150 μL Blocker A to all wells of the MSD plate. | |
| □ 09 | Cover the MSD plate and incubate at room temperature and 500 | |
| | rpm for 1 hour. | |
| □ 10 | Dilution 1: | 1 |
| | Add 490 μL (1) HiSpec assay diluent in micronic tubes 1 | |
| | Add 10.0 μL (2) sample. | 2 |
| | Mix by vortexing. | |
| □ 11 | Dilution 2: | 1 |
| | Add 380 μL (1) HiSpec assay diluent in micronic tubes 2 | |
| | Take 20.0 μL (2) diluted samples from micronic tubes 1 and | 2 |
| | add to the second micronic tubes. | |
| | Mix by vortexing. | |
| □ 12 | Wash the MSD plate 5 times with wash buffer (300 μL/well) and | |
| | tap the plate on a tissue. | |
| □ 13 | Transfer 50.0 μL sample from the micronic tubes 2 in duplicate in | |
| | the appropriate well of the MSD plate. | |
| □ 14 | Cover the MSD plate and incubate at room temperature and 500 | |
| | rpm for 1 hours. | |
| □ 15 | Prepare the detection solution (0.200 μg/mL). Prepare within 15 | 1 |
| | minutes before use. | |
| | Pre-dilution: | 2 |
| | Add 10.0 μL (1) of goat anti-human antibody Sulfo-Tag (17- | |
| | 1422; stock 500 μg/mL) to 90.0 μL (2) HiSpec assay diluent | |
| | and mix by inverting. | |
| | Detection solution: | 3 |
| | Take 6.00 mL (3) HiSpec assay diluent buffer. | |
| | Take out 24.0 μL (2) of the HiSpec assay diluent. | |
| | Add 24.0 μL (2) of the pre-dilution to the HiSpec assay | |
| | diluent and mix by vortexing. | |
| □ 16 | Wash the MSD plate 5 times with wash buffer (300 μL/well) and | |
| | tap the plate on a tissue. | |
| □ 17 | Add 50.0 μL detection solution to all wells of the MSD plate. | |
| □ 18 | Cover the MSD plate and incubate at room temperature and 500 | |
| | rpm for 1 hour. | |
| □ 19 | Prepare the Read Buffer T (2x). Prepare within 15 minutes before | |
| | use. | |
| | Take 9.00 mL of Milli-Q water | |
| | Add 9.00 mL of Read Buffer T (4x) and mix by inverting. | |
| □ 20 | Wash the MSD plate 5 times with wash buffer (300 μL/well) and | |
| | tap the plate on a tissue. | |
| □ 21 | Add 150 μL of Read Buffer (2x) to all wells of the plate. | |
| □ 22 | Measure the response within 5 minutes after the read buffer was | |
| | added to the plate. | |

### Example 3 Characterization of Humoral Immune Response to Met-Var24 Immunization

### Antibody Response in Tg2576 Mice

This study analysed induction of abeta-specific antibody responses in Tg2576 mice immunized monthly with 100 μg Met-var24 from the age of 5 to 16 months. The occurrence of Met-var24-induced abeta-specific antibodies was analysed using a capture ELISA based on immobilization of Aβ1-40.

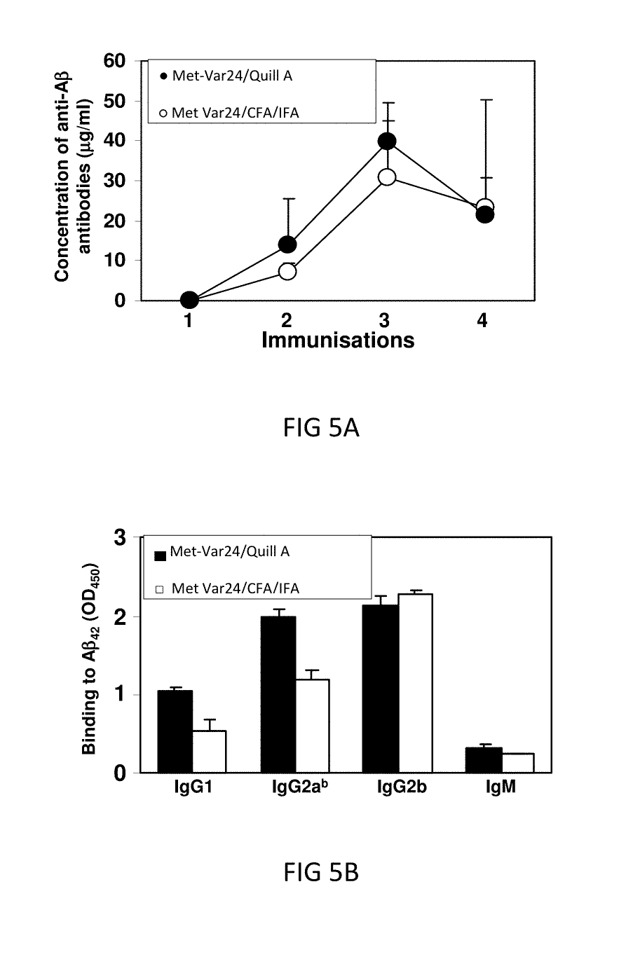

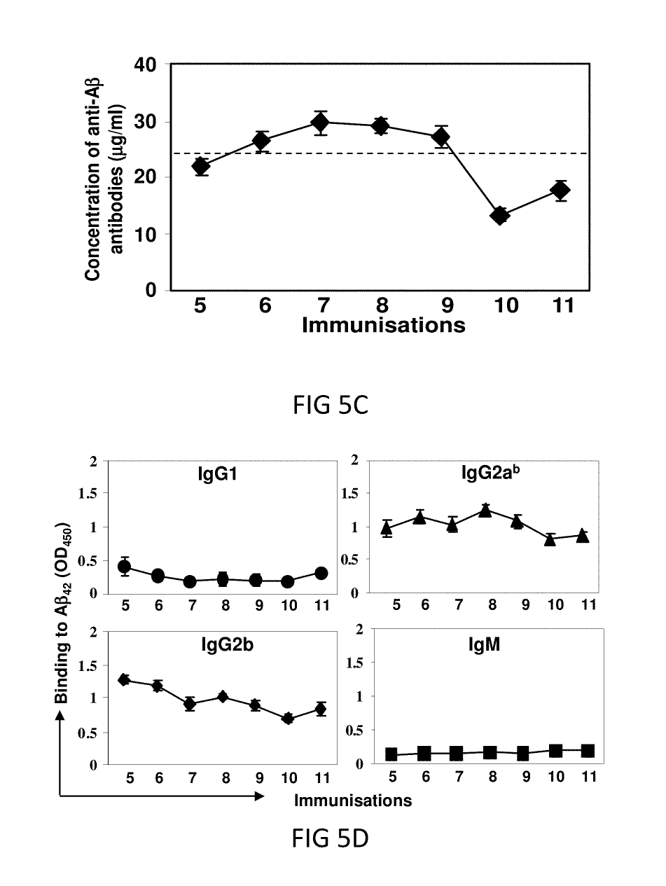

Abeta-specific antibodies were detectable after 2 immunizations (FIG. 5, panel A) and reached a plateau after the 3rd immunization followed by a slight decline after the 4th immunization. No anti-abeta antibodies were detected in the sera of control mice or in pre-immune sera of Met-var24-treated animals.

Immunization with Met-var24 in Tg2576 mice generated antibodies predominantly of the IgG2a and IgG2b isotype (FIG. 5, panel B) with only minor levels of IgM. Also, the Ig subtype composition of Met-var24-induced antibodies remained unaltered during repeated immunizations (FIG. 5, panel D).

### Example 4 Plaque Development in Tg2576 Mice after Immunization with Met-Var24

The effect of Met-var24 on development of abeta plaques were analysed in Tg2576 mice. After 11 immunizations, given monthly, the Tg2576 mice were sacrificed at an age of 17 months and analysed for abeta plaque load. Image analysis of hemibrains stained with anti-MHC II for detection of microglia and with anti-glial fibrillary acidic protein (GFAP) for detection of astrocytes was conducted.

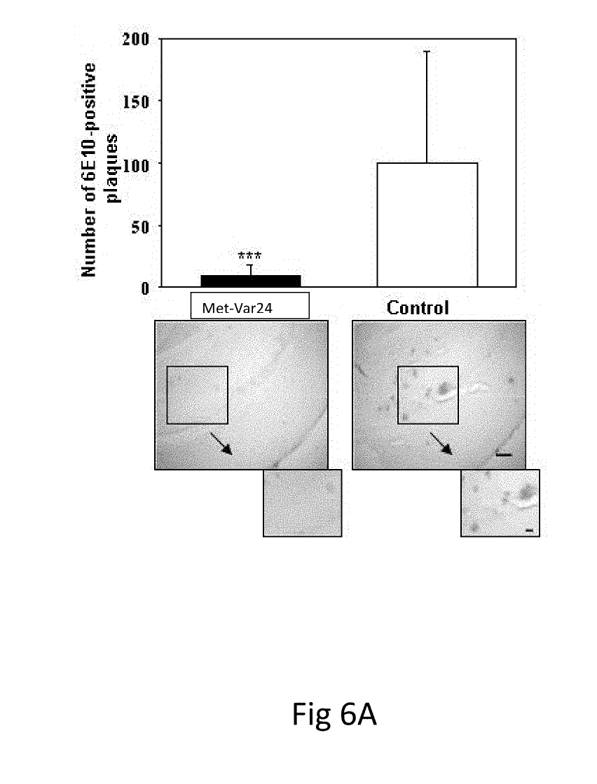

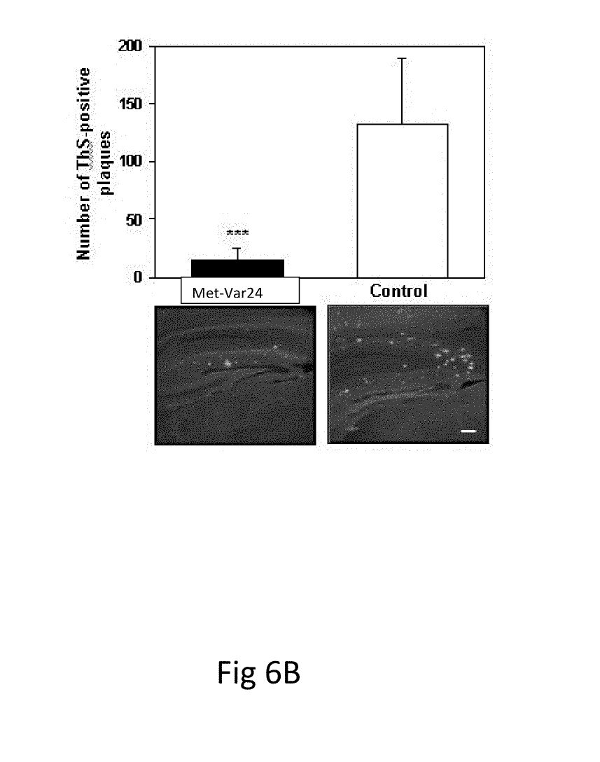

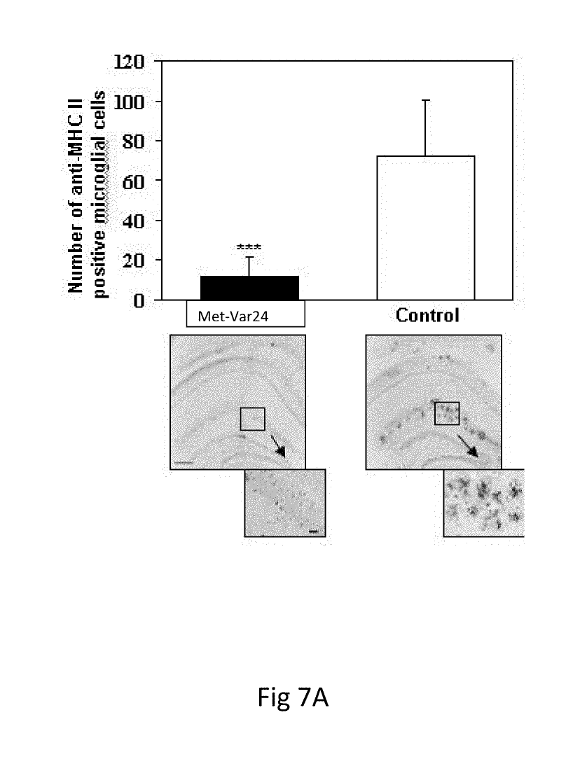

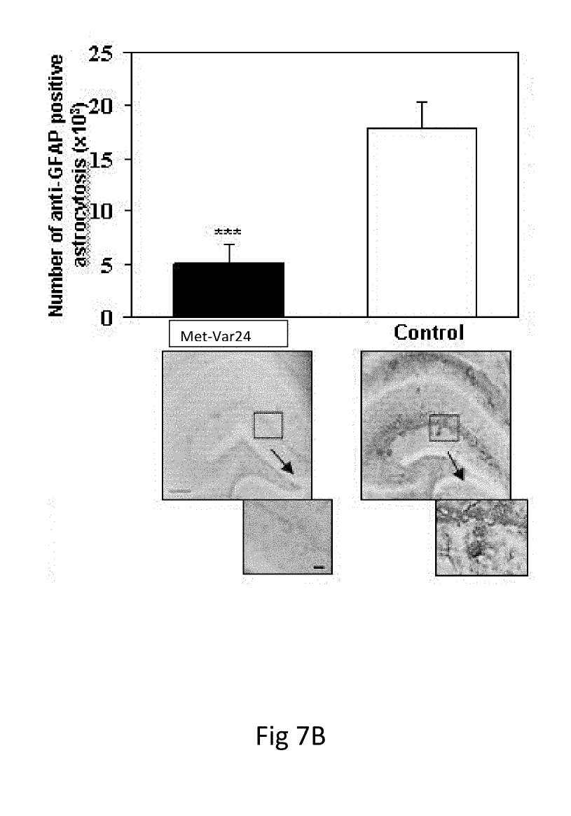

As shown in FIG. 6, immunization with Met-var24 reduced the number of 6E10-positive ((6E10 is commercially available antibody binding to amino acid residue 1-16 of beta amyloid) FIG. 6 A and Thioflavin S (ThS)-positive Aβ plaques (ThS binds to beta sheet-rich structures, (FIG. 6 B). In addition, the Met-var24-immunized mice showed significantly lower activation of microglia and astrocytes in comparison with control Tg2576 mice immunized with adjuvant alone. Importantly, there were no significant differences in cerebral amyloid angiopathy (CAA) and in microbleeds between Met-var24-immunized and control animals (FIG. 7).

In conclusion, Tg2576 mice immunized with Met-var24 showed lower abeta plaque load, less activation of microglia and astrocytes, and no increase in microbleeds compared to controls.

### Binding of Met-Var24-Induced Antibodies to Human Abeta Plaques

The study objective was to investigate if Met-var24-induced antibodies also recognize physiological and pathological relevant abeta. Human AD brain sections were used as source for physiological abeta to verify binding of Met-var24-induced antibodies to disease relevant abeta plaques. Antibodies in plasma from Met-var24-immunized Jucker (APPPS1-21) mice and Cynomolgus monkeys were used.

Paraffin-embedded brain sections from two AD patients were incubated with plasma samples from Met-var24 immunized Jucker (APPPS1-21) mice. Specificity of the plaque binding antibodies for abeta was demonstrated by pre-incubation of the Jucker (APPPS1-21) mouse plasma with increasing concentrations of Met-var24 (0-1000 nM) and the N-terminal abeta amino acids 1-28 (abeta 1-28) (0-1000 nM) before incubation on cortical brain sections. Plasma from Met-var24-immunised Cynomolgus monkeys was diluted 1:200 prior to incubation on cortical brain sections.

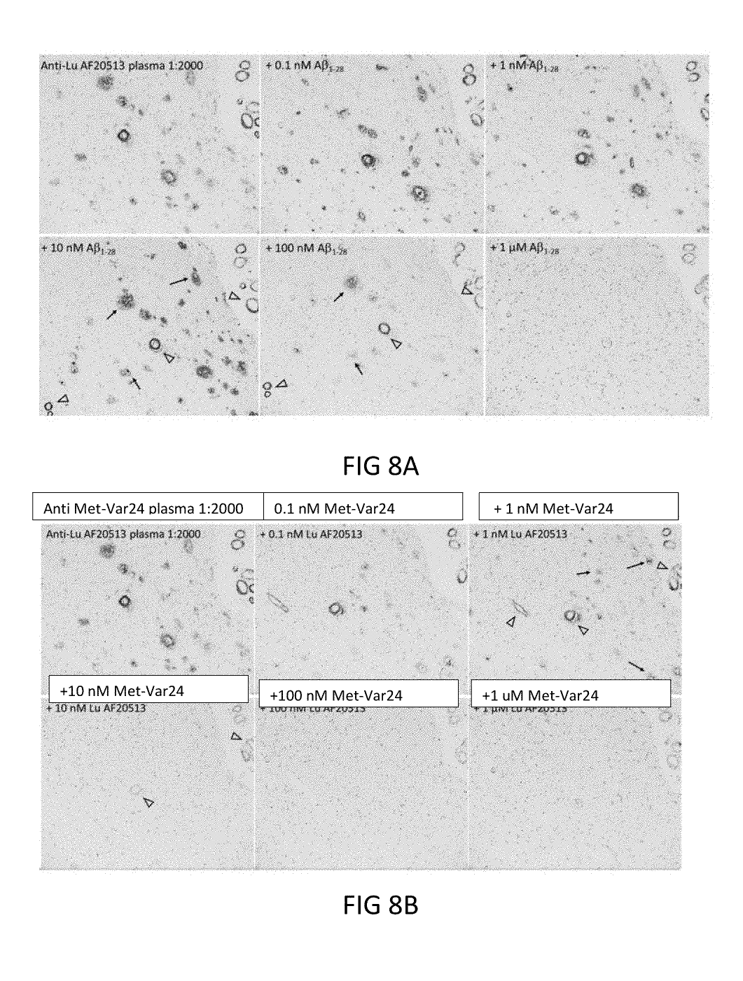

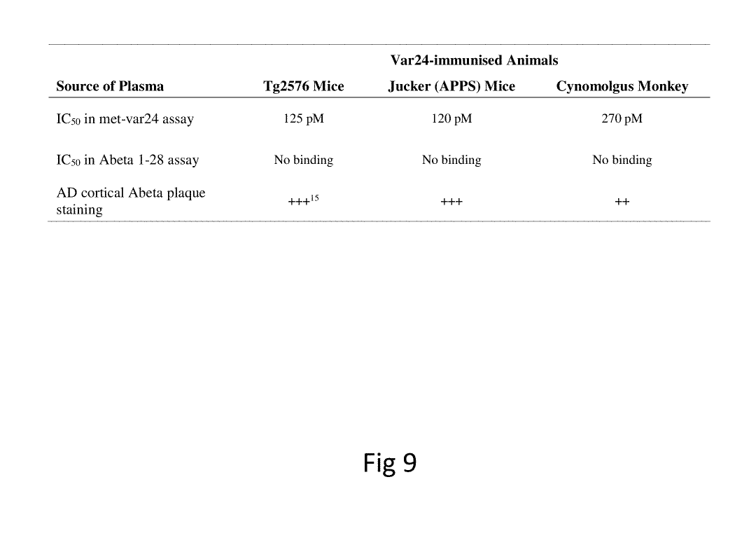

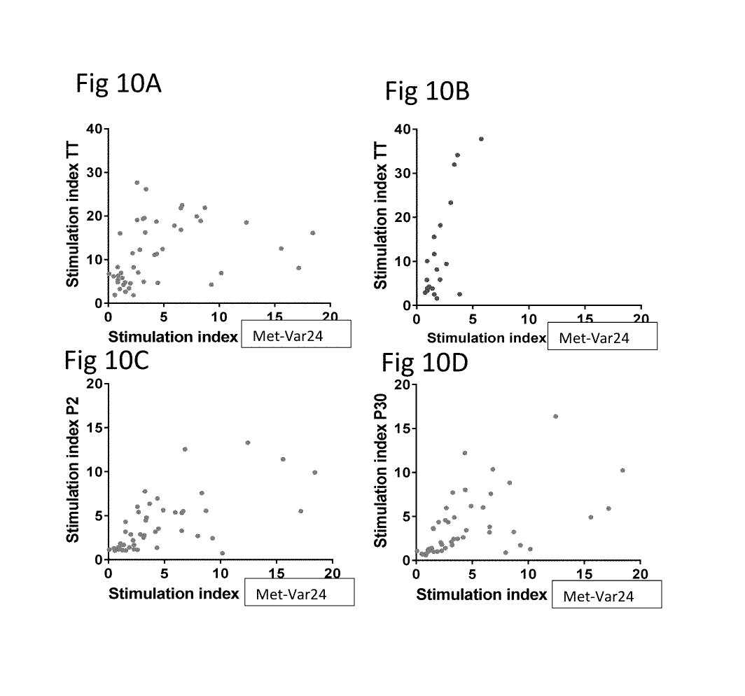

Plasma samples from the Met-var24-immunised Jucker (APPP1-21) mice enabled detection of abeta plaques as shown in FIG. 8. Antibody binding to abeta plaques was abolished completely by pre-incubation of plasma with either 1000 nM abeta 1-28 (FIG. 8A) or 10 nM Met-var24 (FIG. 8B), respectively. The observed binding potencies correspond to the observed binding potency of Met-var24 to Met-var24-induced antibodies. Plasma from immunised Cynomolgus monkeys also demonstrated binding to cortical AD plaques (FIG. 9).

In conclusion, the plaque staining capability of the Met-var24 induced antibodies from immunised animals supports that Met-var24 can elicit an immune response that recognize pathological relevant forms of abeta.

Plasma from Met-var24-immunized animals show binding of pathological abeta which is inhibited by pre-incubation with 10 nM Met-var24 and 1000 nM abeta1-28. Jucker (APPPS1-21) mice primed with P2/P30 peptides prior to Met-var24 immunization received 4 repeating immunisations, one every 4th week from 8 weeks of age. Plasma from bleed 4 (2 weeks post 4th immunization) was pooled (N=10) and diluted 1:2000 stained AD cortex plaques and vacular abeta whereas pre-immune plasma did not.

### Example 5 Effect of Tetanus Peptide Specific Memory T Cells on Met-Var24 Induced Immune Response

The impact of the presence of tetanus peptide specific memory T cells was addressed in 6-8 weeks old B6SJL mice with H-2 genotype identical to Tg2576. These non-transgenic mice were used in this experiment to address induction of memory T cells and the kinetics of T cell response after immunization with Met-var24 given 6 months later.

Two groups of B6SJL mice were immunized with 3 doses given monthly of either P30/Quil-A (50 μg/mouse) or Quil-A alone and subsequently analysed for T memory cells specific to P30 after treatment free period of six months. The memory T cell response was analysed by ELISPOT assays in 10 mice from each group before and after one single Met-var24 (100 μg) injection. CD4+ T cells were purified from splenocytes and responses were measured following in vitro stimulation using P30 and Met-var24 peptides. The purity of isolated CD4+ T cells was verified by flow cytometry. Both cellular and humoral immune responses in Met-var24 treated and control mice were analysed.

In vitro re-stimulation with P30 peptide of splenocytes from P30-immunized B6SJL mice, but not from control splenocytes, induced a significant IFNγ response indicating the presence of T cells responding to P30. Depletion of CD4+ T cells from primed splenocytes completely abrogated detection of IFNγ producing cells. Moreover, enrichment of splenocytes from non-immunized mice with CD4+ T cells, purified from splenocytes from P30 immunized mice restored the P30 stimulation. These observations suggest a population of memory T cells, specific to the tetanus-derived P30 epitope, was present six months after immunization with P30 peptides.

The impact of the P30 memory T cell population on the Met-var24-induced abeta-specific antibodies was subsequently investigated. The abeta-specific antibody response in P30 pre-immunized mice after one single immunization with Met-var24 was found to be 15-fold higher than antibody responses in un-primed mice 15. This observation correlates with the reported observation of an enhanced abeta-specific antibody response after a single Met-var24 immunization of 11 months old Tg2576 mice with P30 specific immune memory after priming with P30 peptide, following a similar procedure for priming as described for B6SJL mice above.

### Example 6 Responses to Met-Var24, P2 and P30 Peptides in Human Peripheral Blood Mononuclear Cells (PBMC)

The objective of a study was to describe the human immunogenic potential of Met-var24, as well as the tetanus peptides P2 and P30 using human PBMCs in vitro stimulation. PBMCs were collected from 52 young (average age: 31 years) and 21 elderly (average age: 70 years) healthy human donors with typical Caucasian Class II HLA allotype frequency distribution. The immunogenicity of Met-var24, P2, P30 and tetanus protein was measured as proliferation of the PBMCs. The stimulation index (SI) describes the degree of proliferation (measured by thymidine incorporation) in antigen-stimulated versus non-stimulated PBMCs. T-cell activation was subsequently studied using flow cytometry that allows differential cell population analyses.

The majority of young healthy Caucasian donors had significant T cell responses to Met-var24, P2 and P30. Met-var24 has a significant immunogenicity as defined by SI values higher than two in 69% of the 52 young donors tested (FIGS. 10 A and C-D). These responders exhibited mostly high SI values ranging from 2.0 to 68.2 (median=4.7). In comparison, P2 and P30 induced significant immune responses in 63% and 60% of the donors tested.

Met-var24 also induced significant immune responses in 38% of the elderly donors tested (8 out of 21 donors (FIG. 10 B). These responder exhibited mostly high SI values ranging from 2.1 to 5.8. P2 and P30 induced immune responses in 38% and 19% of the donors tested (8 out of 21 donors and 4 out of 21 donors, respectively).

### Example 7 First-in-Human (FIH) Study in Patients with Mild AD

### Study Design and Patient Disposition

A currently ongoing and not finalized FIH study consists of two parts, A and B, each including 4 immunizations of Met-var24. In Part A, the duration of patient participation is 48 weeks, including a treatment period of 24 weeks and a follow-up period of 24 weeks. During the treatment period, all patients receive an immunization of Met-var24 at Weeks 0, 4, 12 and 24.

Part B consists of a run-in period of up to 9 months (until the safety data from Part A had been evaluated), followed by a treatment period of up to 36 weeks and a follow-up period of 12 weeks. In Part B, the patients receive an immunization of Met-var24 every 12 weeks. The patients in Cohort 4 participate in Part A of the study (4 immunizations), whereas patients in Cohorts 1 to 3 receive a total of 8 immunizations in Parts A and B.

The patients currently receive the following doses of Met-var24 in Parts A and B:
Cohort 1: Met-var24 Part A: 5 μg, Part B: 50 μg, 10 patients
Cohort 2: Met-var24 Parts A and B: 50 μg, 10 patients
Cohort 3: Met-var24 Parts A and B: 250 μg, 15 patients
Cohort 4: Met-var24 Part A: 2×250 μg, 15

A total of 48 patients, aged 60 to 82 years, had been enrolled into the 4 dose. Thirty-four out of 35 patients completed Part A (1 patient was withdrawn) and 28 patients continued into Part B of the study.

Three patients enrolled in Cohort 3 (250 μg Met-var24) were still currently ongoing in Part B. In addition, Cohort 4 (2×250 μg Met-var24) was still ongoing; 13 out of 15 planned patients had been enrolled and had received up to 3 immunizations of Met-var24.

### Antibody Titre

One of the main objectives was to examine the time course and magnitude of the abeta-specific antibody response following multiple immunizations with Met-var24 in humans.

The antibody data from Cohorts 1 to 3 (Part A only), obtained using two methods for assessment of abeta-specific antibodies, suggest a dose-response relationship with a higher number of responders in the 250-m cohort compared to the lower doses of 5 and 50 μg Met-var24. A titre responder is defined as any patient with a sample taken at any timepoint after immunization with a titre above the assay cut point as described for the current invention.

The number of antibody titre responders in Cohorts 1 to 3 (Part A only), based on the results from two assays, are summarized the below table.

TABLE II
Antibody Titre Responders in Cohorts 1 to 3 - Part A

Responders

| Treatment Group | N | n | % | 95% CI |
|---|---|---|---|---|
| Part A: 5 μg | 9 | 6 | 66.7 | (35.4, 87.9) |
| Part A: 50 μg | 10 | 8 | 80.0 | (49.0, 94.3) |
| Part A: 250 μg | 15 | 15 | 100.0 | (79.6, 100.0) |

Titre responder: any patient with a sample taken at any timepoint after immunization with a titre above the assay cut point as described for the current invention and above the patient's baseline titre in the applied assays for abeta-specific antibody assessment.
ATAS_A: antibody-titre-analysis set in Part A
Wilson's score-type confidence intervals are derived for the titre response rates.

1. A method of treating a patient having Alzheimer's disease (AD) by treating said patient with an effective amount of Met-var24 or X-var24.

2. The method according to claim 1, wherein the patient is Caucasian.

3. The method according to claim 1, wherein the treatment is done by subcutaneous or intramuscular administration.

4. The method according to claim 1, wherein the patient has received a tetanus toxoid vaccine prior to receiving said effective amount of Met-var24 or X-var24.

5. The method according to claim 1, wherein the patient has received vaccine comprising P2 and/or P30 from tetanus toxoid prior to receiving said effective amount of Met-var24 or X-var24.

6. The method according to claim 1, wherein an evaluation of the treatment response of the treatment is done by evaluating the induced titre or IC50.

7. The method according to claim 6, wherein an evaluation of the treatment response is made after the patient has been treated 1, 2, 3, 4 or more times.

8. The method according to claim 6, wherein said evaluation is made in an immunoassay comprising the steps of
a. Applying a sample obtained from a patient according to claim 1 in an immunoassay coated with Met-var24 under appropriate binding conditions,
b. Measuring the antibody titre or IC50.

9. The method according to claim 6, wherein immune therapy of the patients are continued if the titre and/or IC50 shows that a beneficial antibody response is obtained in the immunized patient.

10. The method according to claim 6, wherein immune therapy of the patients is discontinued if the titre and/or IC50 shows that a beneficial antibody response is not obtained in the immunized patient.

11. The method according to claim 6, wherein the immunoassay is an ELISA or MSD assay.

12. The method according to claim 11, wherein a titre above 1.000 indicates a beneficial antibody response.

13. The method according to claim 6, wherein an IC50 of in the range of about 200 to about 1000 pM indicates a beneficial antibody response.

14. The method according to claim 1, wherein the patient receives a dose of 50 μg, 100 μg, 250 μg, 500 μg or 1000 μg Met-var24 or X-var24.

15. The method according to claim 1, wherein the patient receives a dosage regime of 2 simultaneous dosages of 250 μg Met-var24 or X-var24.

16. The method according to claim 1, wherein treatment is done every 2 week, every 4 week or every 6 week.

17. A method of treating a patient having Alzheimer's disease by treatment with an effective amount of Met-var24 or X-var24, wherein said patient upon earlier treatments with Met-var24 or X-var24 has been shown to raise a titre response according to claim 1.

18-33. (canceled)

34. A composition comprising Met-var24 or X-var24 as defined by SEQ ID NO:2 or 3, respectively.

35. A pharmaceutical composition comprising Met-var24 as defined in claim 34 as the only immunogenic peptide in a purity of 90% such as 95%, 98% or 100%.

36. An in vitro method for quantification of antibodies from an Alzheimer's disease patient comprising the steps of
a) Taking a sample from a patient, and
b) Analyzing the sample in an immunoassay.

37-56. (canceled)
